(19) United States
(12) Patent Application Publication
(10) Pub. No.: US 2024/0326119 A1
(43) Pub. Date: Oct. 3, 2024

(54) METHOD FOR THE ADDITIVE MANUFACTURING OF CASTING MOLDS

(71) Applicants: ETH ZÜRICH, Zürich (CH); TECHNISCHE UNIVERSITEIT DELFT, Delft (NL)

(72) Inventors: André R. STUDART, Zürich (CH); Kunal MASANIA, Oegstgeest (NL); Nicole KLEGER, Weiningen (CH); Simona FEHLMANN, Zürich (CH); Martina CIHOVA, London (GB)

(21) Appl. No.: 18/580,730

(22) PCT Filed: Apr. 28, 2022
(86) PCT No.: PCT/EP2022/061334

§ 371 Date: Jan. 19, 2024

(30) Foreign Application Priority Data
Jul. 20, 2021 (EP) 21186780.9

(51) Int. Cl. B22C 9/10; B22C 1/10; B22D 18/06; B22D 29/00; B29C 33/38; B29C 33/52; B29C 33/76; B29C 39/26; B29K 63/00; B33Y 10/00; B33Y 40/20; B33Y 80/00; C09D 11/037; C09D 11/101; C09D 11/107

(52) U.S. Cl. CPC B22C 9/105; B22C 1/10; B22D 18/06; B22D 29/002; B29C 33/3842; B29C 33/52; B29C 33/76; B29C 39/26; B33Y 10/00; B33Y 80/00; C09D 11/037; C09D 11/101; C09D 11/107; B29K 2063/00; B29K 2909/02; B33Y 40/20

(57) ABSTRACT

A method for manufacturing a photopolymerizable slurry comprises the steps of: providing a plurality of particles of an inorganic salt in the form of a powder; wherein at atmospheric pressure the inorganic salt has a melting point of above 250° C.; and wherein at room temperature the inorganic salt has a solubility in water above 9% w/w; providing at least one radiation curable monomer; the at least one radiation curable monomer being in the liquid phase; and adding the inorganic salt particles to the liquid composition and mixing the inorganic salt particles with the liquid composition; obtaining a photopolymerizable slurry, selectively curing the photopolymerizable slurry to obtain a green body article; debinding the green body article to obtain a binderless body article; and sintering the binderless body article to obtain a sintered ceramic article; providing a first template mold (1); wherein the first template mold comprises the sintered ceramic article; providing a second mold (2, 2′, 2″); wherein the second mold comprises a compartment into which said first template mold can be placed; mounting the first template mold into the compartment of the second mold, thereby obtaining an operative mold (1, 2, 2′, 2″); casting a fluid casting material (3, 3′, 3″) into said operative mold to obtain after solidification of said casting material an infiltrated template mold (8) comprising a solid article (9) that is at least partially located within the first template mold (1); and separating said solid article from the first template mold by dissolving the sintered ceramic article of the first template mold with a suitable solvent, for example water.

### FIELD OF THE INVENTION

The invention relates to photopolymerizable slurries, methods for manufacturing such photopolymerizable slurries, methods for manufacturing sintered ceramic articles with such photopolymerizable slurries, and a method for casting articles.

### BACKGROUND OF THE INVENTION

Shaping of matter into complex structures has been greatly advanced through the advent of additive manufacturing techniques. A crucial development in this field has been the design and formulation of resins or inks with a broad variety of material compositions [11, 24, 35, 36]. This has turned 3D printing from a prototyping tool into a manufacturing platform for functional objects and devices in health [37], energy [38], architecture [39], and robotic applications [40]. Using inks with self-assembling building blocks has expanded the capabilities of 3D printing techniques to the fabrication of structures with intricate hierarchical architectures and feature sizes below accessible printer resolution [11, 41, 42, 43, 44]. Despite these enticing achievements and promising prospects, many materials are still not directly 3D printable due to challenges in fulfilling the rheological, physical and chemical requirements of extrusion- and light-based printing techniques [45]. In addition, the lack of reproducibility and reliability of additive processing has made it difficult to produce parts for critical applications such as aerospace and medical, where certification of microstructure and part integrity is paramount.

The manufacturing of structures with controlled chemical composition, complex shape and porosity is crucial for many applications in medicine, engineering, and architecture. Because shaping technologies are not always available for the materials/chemical compositions of interest, it is sometimes convenient to manufacture templating molds into which materials with desired chemistry can be cast. Additive manufacturing has been extensively employed to fabricate such complex-shaped molds. However, removal of the mold after casting might be challenging or impossible when the final articles feature complex shapes or internal porosity.

3D printed sacrificial molds and templates offer a universal approach to shape matter into complex architectures without rheological or chemical modification of the material of interest. The idea of such indirect additive manufacturing is to first print the sacrificial template into the desired negative geometry and then cast or deposit the material of interest before final removal of the template [22, 46-48,49-52]. This manufacturing strategy has enabled the fabrication of intricate structures from a wide range of materials at very different length scales. At the centimeter scale, binder-jetting printed sand or stereographically printed polymer molds have been utilized to fabricate complex metal parts by investment casting [22,52] and elastomer parts by casting silicone resins, respectively [48,50]. At the millimeter scale and below, two-photon polymerization has been exploited to print three-dimensional polymer templates for the creation of metal and ceramic micro-lattices with exquisite architectures and mechanical properties [53,54]. In many of these processes, the template material needs to be dissolved with chemicals or thermally degraded at temperatures above 400° C. [55,56]. This often makes the process energy demanding and/or increases the probability of cracking or deformation of the templated material.

Porosity is an essential feature in a wide range of applications that combine light weight with high surface area and tunable density. Porosity is desired in a broad range of materials for applications such as catalytic supports and lightweight structures. Particularly, porous materials are of high interest in the biomedical field, especially for their use in tissue engineering and bioresorbable implants, such as implants for osteosynthesis.

One approach to manufacture porous casts in shown in CN 110407604. Gypsum is cast into a sponge, and after solidification of the gypsum cast the sponge is burned out, resulting in a lost mold that can then be used to cast a porous article. Similar methods are known from U.S. Pat. Nos. 3,616,841 and 3,946,039. Slurries used in said methods for producing the ceramic lost mold do not comprise a binding agent.

Highly porous materials can be obtained by several approaches such as salt leaching, gas foaming, freeze-drying and sintering, and phase separation [1]. A cost-effective and sustainable strategy to circumvent the above-mentioned processing issues is to use readily soluble inorganic salts such as sodium chloride (NaCl), as molds or templates. Because NaCl is non-toxic and readily dissolvable in water at ambient temperature, salt templates do not require excessive heating or toxic solvents to be removed and are broadly studied for biomedical applications. These features also make them suitable templates for shaping temperature-sensitive materials, such as living materials, hydrogels and polymers. Alternatively, the physical and mechanical stability of NaCl up to its melting temperature of 801° C. enables infiltration of materials at elevated temperature in the molten state. Such versatility has allowed for the use of salt particles as pore-forming templates for the fabrication of a broad range of materials, from aluminum foams for structural applications [6] to porous hydrogels for tissue engineering [57,58]. With the salt leaching technique, porous materials based on all material classes, i.e. metals, ceramics or polymers, can be obtained [2]. Typically, salt particles are used as leachable powder to produce a compacted preform, which is then infiltrated with the material of interest. Upon solidification of the infiltrate, the salt is removed by leaching in a suitable solvent, and the solidified, infiltrated material remains. The salt leaching technique has been used to achieve porosity in a wide variety of materials, including natural polymers such as silk fibroin [3], synthetic polymers such as poly(l-lactic acid) [4], and bulk metallic glasses [5], and crystalline metals such as aluminum [6] and magnesium [7]. In all these approaches, the pore size of the final part is defined by the size of the original salt particles, or the salt particle aggregates used as the template. The pore geometry of the resulting material is random, with broad pore-size distributions, which reflects the polydisperse nature of the salt particle template matrix. This limits this technique's ability to control the porous architecture and achievable overall porosity of the final scaffold.

Beyond templating particles, salt has also been deposited around 3D printed polymeric templates to create castable molds upon polymer removal [22,52]. The direct printing of NaCl into three-dimensional grid-like structures for the fabrication of magnesium with unique porous architectures has been described [22]. However, 3D printing of NaCl for extrusion-based direct ink writing is limited in both the attainable resolution and the freedom of architectural design. Novel approaches to manufacture complex-shaped salt templates are highly demanded.

Additive manufacturing allows to manufacture complex three-dimensional shapes and is also used in the manufacturing of porous materials, where it allows tuning of the pore geometries and sizes. Additive manufacturing techniques allow to produce three-dimensional structures with well-controlled porosity and pore sizes from nanoscale to macroscale [8, 9, 10, 11, 12]. Various additive manufacturing techniques such as selective laser sintering (SLS), fused deposition modeling (FDM), selective laser melting (SLM) or stereolithography (SL) have been applied. SLS and FDM have been mainly applied for polymeric materials and as a combination with ceramics, e.g. polycaprolactone (PCL), polylactic acid (PLA), hydroxyapatite (HA), and calcium phosphate (CaP). SLM has been used for the fabrication of metal parts from metal powder, e.g. Mg. Moreover, porous parts from PCL and CaP were also achieved with stereolithography printing. [13,14,15]

While there is a broad choice of materials available for additive manufacturing, it is difficult to find materials that are both suitable for common AM techniques and result in a product having a mechanical strength suitable for the end application (e.g. as bone replacement) and still being biodegradable. It also remains a challenge to shape materials that possess a high chemical reactivity, such as for example magnesium, using additive manufacturing techniques.

Magnesium (Mg) is receiving increasing attention as a metallic biodegradable implant material for temporary bone replacement or osteosynthesis [16]. Magnesium has similar mechanical properties as bone and is able to induce new bone formation [17] and is bioresorbable [18]. It is widely accepted that pore size, pore shape, pore directionality, and the degree of porosity of Mg scaffolds are factors that strongly influence cell viability and growth. To guide bone-tissue growth, large open porosity with pore sizes >300 μm in combination with surface roughness appears to be most successful. [19, 20] Thus, the ability to shape the magnesium into structures with controlled porosity and pore size, and in a patient-specific outer geometry, is highly desired.

A three-step process has been reported for indirect additive manufacturing of metallic magnesium [21, 22]. A 3D polymer structure is produced via AM. The resulting polymer structure is then infiltrated with a NaCl paste. Upon removal of the polymer structure, the NaCl acts as a template for Mg infiltration. In this technique, the additional processing step required to generate the polymer template increases the processing time and results in enhanced risk of imperfect structure replication. Furthermore, the technique is limited to geometries that allow the necessary infiltration of the highly viscous NaCl paste into the polymer template.

WO 2020/046687 A1 discloses a method of making a non-oxide ceramic part. A photopolymerizable slurry, containing non-oxide ceramic particles with high melting point such a silicon carbide or boron nitride, is selectively cured to obtain a gelled article. The gelled article is dried to form an aerogel article or a xerogel article. The aerogel article or xerogel article is heat treated to form a porous ceramic article, which is then sintered to obtain a sintered ceramic article.

N. Kleger et al. [23] describe a method for directly 3D printing a NaCl template for Mg infiltration, using an ink consisting of NaCl particles dispersed in paraffin oil. In this method, a three-dimensional structure with controlled pore geometry can be printed via the direct ink writing technique. After removal of the paraffin oil, the resulting green body is calcined and sintered to a NaCl template. The template is infiltrated with melted magnesium. After leaching the NaCl template structure, the solid magnesium scaffold remains. The spatial resolution of the disclosed method is limited. Moreover, mechanical stability issues of the green body limit the available geometries.

Manufacturing processes used for the fabrication of inorganic materials often involve the addition to organic binders to the inorganic particles of interest and the formation of a green body that is later subjected to debinding and sintering at high temperatures. A common problem when debinding green bodies consisting of organic binder and ceramic particles is crack formation. While debinding is thought to be a common cause of cracking, polymerization shrinkage, low particle concentrations and the presence of unreacted monomer can also lead to the formation of cracks during the manufacturing process [24, 25, 26, 27]. No general solution has been found so far to avoid such crack formation.

When such leachable structures are intended to be used as templates, the infiltration of the templates with the material of interest is an important step. There exists a variety of techniques for infiltration, such as pressure and vacuum infiltration, chemical vapor infiltration, and injection molding. Infiltration processes are also commonly used to fabricate composite materials.

For the production of metal matrix composites (MMC), mostly vacuum infiltration is used. Typically, a porous preform and the metal to be infiltrated are first evacuated in an oven before the metal melts. Then, pressurized, inert gas is filled into the oven, forcing the molten metal into the preform. The surface tension of the metal and its wettability on the preform material play an important role for the quality of the final product.

Chemical vapor infiltration works similar to the aforementioned infiltration, however the infiltrate is a reactant gas flowing through the preform (mixture of carrier gas along with the matrix material of interest). This is achieved through diffusion or a pressure difference. The matrix material reacts chemically with the preform and hence results in a composite material.

Injection molding is most popular for the processing of polymeric material but can also be used for metals and ceramics. Typically, the molten cast material is injected under high pressure into a heated mold. After cooling, the molded part is removed from the mold. The mold contains small air vents, placed at appropriate places in the mold wall, which allow the air to escape when the material is injected. Injection molding is not an infiltration technique per se but can be used as such. Vacuum pressure infiltration as well as injection molding are both techniques that can be used for the infiltration of sintered ceramic articles.

There is a general need for improvements in this field.

### SUMMARY OF THE INVENTION

It is the overall objective of the present invention to provide improvements in manufacturing cast objects with template molds.

One object of the present invention is to provide advantageous photopolymerizable slurries that allow to manufacture molds made of soluble inorganic salts, with higher spatial resolution and/or increased mechanical stability.

Another object of the invention is to provide advantageous methods for manufacturing such photopolymerizable slurries, methods for manufacturing sintered ceramic articles made of soluble inorganic salts, as well as methods for manufacturing cast articles.

These and other objects are substantially achieved through the features of the independent claims. Advantageous embodiments and variants are set forth in the dependent claims.

Further aspects of the present invention become evident as this description proceeds.

The present invention may comprise one or more of the features recited in the attached claims, and/or one or more of the following features and combinations thereof.

One aspect of the invention concerns a photopolymerizable slurry.

A photopolymerizable slurry according to the invention comprises a plurality of particles of an inorganic salt and at least one polymerizable monomer or oligomer. The cation of the inorganic salt is a metal cation. The anion of the inorganic salt is neither oxide nor hydroxide. The inorganic salt has a melting point of above 250° C. at atmospheric pressure and has a solubility in water above 9% w/w at room temperature.

Metal oxides and metal hydroxides are not suitable for a photopolymerizable slurry according to the invention, particularly due to their highly caustic properties, which can negatively affect the intended use of the photopolymerizable slurry, for example the polymerization reaction.

The term “polymerizable monomer or oligomer” shall comprise molecules that can react together with other monomer or oligomer molecules to form a larger polymer.

The term “photopolymerizable” shall refer to the ability of a chemical composition to a polymerization reaction that requires actinic radiation (e.g. UV or visible light) for the propagation step.

As used herein, the term “polymerizable slurry” means a mixture of solid material suspended or dispersed in a liquid, of which composition at least one component can undergo polymerization upon initiation, e.g. free-radical polymerization initiation. As a result, the mixture undergoes gelation. Typically, prior to gelation, the polymerizable slurry has a viscosity profile consistent with the requirements and parameters for the additive manufacturing method it is intended to be used for (e.g. 3D printing). Irradiation with actinic radiation having sufficient energy to initiate a polymerization or cross-linking reaction, for instance ultraviolet (UV) radiation or electron beam radiation, or both, can be used for this purpose. By exposing the photopolymerizable slurry to a suitable light source, the radiation-curable monomer or oligomer can be polymerized. The resulting polymer matrix acts as a binder for the inorganic salt particles, resulting in a green body.

The term “inorganic salt” shall comprise salts consisting of inorganic ions, including carbonate and hydrogen carbonate ions.

Advantageously, in a photopolymerizable slurry according to the invention, the anion of the inorganic salt is selected from a group consisting of bromide, chloride, fluoride, iodide, sulfate, nitrate, nitrite, carbonate, and cyanide.

Advantageously, in a photopolymerizable slurry according to the invention, the metal cation of the inorganic salt is selected from a group consisting of barium, beryllium, cadmium, calcium, cesium, cobalt, copper, iron, lead, lithium, magnesium, manganese, nickel, potassium, rubidium, silver, sodium, and zinc.

A melting point above 250° C. of the inorganic salt allows a sufficient increase in temperature to remove the polymer binder and other carbonaceous compounds by pyrolysis, without melting the particles and destroying the binderless body.

For an efficient removal of a template mold after a casting process by dissolving the mold in water or an aqueous solution, the inorganic salt used for producing the mold should have a sufficiently high solubility, above 9% w/w in water.

Inorganic salts that can be used for such a photopolymerizable slurries, and that have a melting point of above 250° C. at atmospheric pressure and a solubility in water above 9% w/w at room temperature are listed Table 1.

TABLE 1
Salts

| | | Melting | Solubility in water |
|---|---|---|---|
| Name | Formula | point [° C.] | [g/100 g water] |
| Barium bromide | BaBr₂ | 857 | 100 |
| Barium chloride | BaCl₂ | 961 | 37 |
| Barium iodide | BaI₂ | 711 | 221 |
| Beryllium chloride | BeCl₂ | 415 | 71.5 |
| Beryllium sulfate | BeSO₄ | 1127 | 41.3 |
| Cadmium bromide | CdBr₂ | 568 | 115 |
| Cadmium chloride | CdCl₂ | 568 | 120 |
| Cadmium sulfate | CdSO₄ | 1000 | 76.7 |
| Calcium bromide | CaBr₂ | 742 | 156 |
| Calcium chloride | CaCl₂ | 775 | 81.3 |
| Calcium iodide | CaI₂ | 783 | 215 |
| Cesium chloride | CsCl | 646 | 191 |
| Cobalt chloride | CoCl₂ | 737 | 56.2 |
| Copper(II) bromide | CuBr₂ | 498 | 126 |
| Copper(II) chloride | CuCl₂ | 598 | 75.7 |
| Iron(II) bromide | FeBr₂ | 691 | 120 |
| Iron(II) chloride | FeCl₂ | 677 | 65 |
| Lead(II) nitrate | Pb(NO₃)₂ | 470 | 59.7 |
| Lithium bromide | LiBr | 550 | 181 |
| Lithium chloride | LiCl | 610 | 84.5 |
| Lithium iodide | LiI | 469 | 165 |
| Lithium nitrate | LiNO₃ | 253 | 102 |
| Magnesium bromide | MgBr₂ | 711 | 102 |
| Magnesium chloride | MgCl₂ | 714 | 56 |
| Manganese(II) bromide | MnBr₂ | 698 | 151 |
| Manganese(II) chloride | MnCl₂ | 650 | 77.3 |
| Manganese(II) bromide | MnBr₂ | 698 | 151 |
| Manganese(II) chloride | MnCl₂ | 650 | 77.3 |
| Nickel(II) bromide | NiBr₂ | 963 | 131 |
| Nickel(II) chloride | NiCl₂ | 1031 | 67.5 |
| Potassium bromide | KBr | 734 | 25 |
| Potassium carbonate | K₂CO₃ | 899 | 111 |
| Potassium chloride | KCl | 771 | 25 |
| Potassium cyanide | KCN | 622 | 69.9 |
| Potassium fluoride | KF | 858 | 102 |
| Potassium iodide | KI | 681 | 148 |
| Potassium nitrate | KNO₃ | 334 | 38.3 |
| Potassium nitrite | KNO₂ | 438 | 312 |
| Potassium sulfate | K₂SO₄ | 1069 | 12 |
| Rubidium chloride | RbCl | 724 | 93.9 |
| Silver fluoride | AgF | 435 | 172 |
| Sodium bromide | NaBr | 747 | 94.6 |
| Sodium carbonate | Na₂CO₃ | 856 | 30.7 |
| Sodium chloride | NaCl | 802 | 36 |
| Sodium cyanide | NaCN | 562 | 58.22 |
| Sodium nitrate | NaNO₃ | 306.5 | 91.2 |
| Sodium sulfate | Na₂SO₄ | 884 | 28.1 |
| Zinc bromide | ZnBr₂ | 402 | 488 |
| Zinc chloride | ZnCl₂ | 325 | 408 |
| Zinc iodide | ZnI₂ | 450 | 438 |

It is also possible to use mixtures of inorganic salts. Interactions of different salt compounds, particularly interactions of particles of different salts, can influence the local and/or the overall behavior of the salt particles, particularly the melting temperatures, and the sintering behavior.

Advantageously, the used inorganic salt consists of ions that are physiologically acceptable. For example, sodium, potassium, calcium, and magnesium can be used as cations, and chloride and carbonate can be used as anions. Sodium chloride (NaCl) and potassium chloride (KCl) are particular advantageous choices for the inorganic salt in view of physiological safety, melting temperature, solubility in water without the need of organic solvents, costs, biocompatibility, high thermal and chemical stability, and applicability to a vast range of scaffold materials.

The polymerization reaction of the monomer in the photopolymerizable slurry can be initiated by irradiation with suitable actinic radiation.

Advantageously, the photopolymerizable slurry can comprise a photoinitiator.

A photoinitiator is a molecule that creates reactive species (free radicals, cations or anions) when exposed to actinic radiation (UV or visible light). The photoinitiator must be suitable for the applied system of polymerizable monomer or oligomer,

By irradiation of a certain volume of the photopolymerizable slurry with light, the polymerization reaction in this volume is initiated, and the photopolymerizable slurry in this volume solidifies.

In an advantageous variant of a photopolymerizable slurry according to the invention, the inorganic salt particles are coated or functionalized with a dispersant.

This surface coating or functionalization of the inorganic salt particles is advantageous in regard to constant and reproducible properties of the slurry and the green body produced from it.

An advantageous variant of a photopolymerizable slurry according to the invention further comprises an inhibitor.

The inhibitor can for example be a UV blocker, a compound that absorbs UV light. This is advantageous in regard to increased spatial resolution during the additive manufacturing process.

The inhibitor can be a chemical additive that inhibits or retard the degradation (oxidation, thermal degradation, etc.) of the polymerizable slurry or the solidified green body. This is advantageous e.g. in regard shelf life, mechanical stability of the polymer matrix during debindering, etc.

An advantageous variant of a photopolymerizable slurry according to the invention further comprises a diluent.

A diluent can be used to adjust the viscosity of a photopolymerizable slurry.

A diluent can also be used to improve the mechanical stability of the green body or the binderless body.

At room temperature, the diluent can be a liquid, or a solid compound that is dissolved in another component of the photopolymerizable slurry, e.g. in a liquid monomer.

Advantageously, camphor is used as the diluent.

An advantageous variant of a photopolymerizable slurry according to the invention further comprises a sintering aid.

Advantageously, the sintering aid improves the sintering step. For example, Na₂SO₄ can be used to decrease the melting point of NaCl used as the inorganic salt.

Another aspect of the invention concerns a method for manufacturing a photopolymerizable slurry.

A method for manufacturing a photopolymerizable slurry according to the invention comprises the steps:
a) providing a plurality of particles of an inorganic salt in the form of a powder; wherein the inorganic salt has a melting point of above 250° C. at atmospheric pressure; and wherein the inorganic salt has a solubility in water above 9% w/w at room temperature;
b) providing at least one polymerizable monomer or oligomer; the polymerizable monomer or oligomer being in the liquid phase; and
c) adding the inorganic salt particles to the liquid composition and mixing the inorganic salt particles with the liquid composition; obtaining a photopolymerizable slurry.

In an advantageous variant of such a manufacturing method according to the invention, the inorganic salt particles are produced by milling an amount of inorganic salt together with a dispersant in a liquid phase, thereby obtaining a dispersion of inorganic salt particles coated or functionalized with the dispersant; and removing the liquid phase.

The milling may for example be carried out in a suitable ball mill.

The liquid phase can be removed for example by evaporation of the liquid phase, or by spray drying.

In an advantageous variant of such a manufacturing method according to the invention, a photo initiator, and/or an inhibitor, and/or a diluent, and/or a sintering aid are provided.

More advantageously, the provided photo initiator, inhibitor, diluent, or sintering aid are in a liquid state, or are solids that are dissolved in the monomer or oligomer, obtaining a liquid composition.

A further aspect of the invention concerns a method for manufacturing a sintered ceramic article.

A method for manufacturing a sintered ceramic article according to the invention comprises the steps:
a) providing a photopolymerizable slurry according to the invention;
b) selectively curing the photopolymerizable slurry to obtain a green body article;
c) debinding the green body article to obtain a binderless body article; and
d) sintering the binderless body article to obtain a sintered ceramic article.

The term “binderless body” designates a green body after debinding, namely the removal of the binder, i.e. the polymer matrix, e.g. by pyrolysis. The removal of the binder matrix can be incomplete. The rest of the binder will burn off during the sintering step.

The remaining polymer can help to connect the inorganic salt particles until in the subsequent sintering step the individual particles are sintered together. Thus, a binderless body may comprise less than 10%, advantageously less than 5%, even more advantageously less than 2% of the original weight of the polymer in the green body.

In an advantageous variant of such a manufacturing method according to the invention, the selective curing of the photopolymerizable slurry to obtain a green body article is carried out within an additive manufacturing process.

Advantageous additive manufacturing methods that can be applied for the additive manufacturing process in such a manufacturing method are for example stereolithography (SLA) 3D printing, 2-photon polymerization (2PP), or digital light projection (DLP) 3D printing.

Another aspect of the invention concerns a green body article as it can be obtained after step b) of a method for manufacturing a sintered ceramic article according to the invention.

Another aspect of the invention concerns a binderless body article, as it can be obtained after step c) of a method for manufacturing a sintered ceramic article according to the invention.

Another aspect of the invention concerns a sintered ceramic article, manufactured with a method for manufacturing a sintered ceramic article according to the invention.

A further aspect of the invention concerns a method for casting articles.

A method for manufacturing cast articles according to the invention comprises
a) providing a first template mold; wherein the first template mold comprises a sintered ceramic article according to the invention;
b) providing a second mold; wherein the second mold comprises a compartment into which said first template mold can be placed;
c) mounting the first template mold into the compartment of the second mold, thereby obtaining an operative mold;
d) casting a fluid casting material into said operative mold to obtain after solidification of said casting material an infiltrated template mold comprising a solid article that is at least partially located within the first template mold; and
e) separating said solid article from the first template mold by dissolving the sintered ceramic article of the first template mold with a suitable solvent, for example water.

Since the first template mold is destroyed in the separation step, the first template mold is a so-called lost mold.

A particularly advantageous variant of such a method for manufacturing cast articles comprises the steps:
providing a first template mold; wherein the first template mold comprises a sintered ceramic article manufactured with a method comprising the steps:
providing a photopolymerizable slurry, comprising a plurality of particles of an inorganic salt; and at least one polymerizable monomer or oligomer; wherein the inorganic salt has a melting point of above 250° C. at atmospheric pressure; and wherein the inorganic salt has a solubility in water above 9% w/w at room temperature;
selectively curing the photopolymerizable slurry to obtain a green body article;
debinding the green body article to obtain a binderless body article; and
sintering the binderless body article to obtain a sintered ceramic article.
providing a second mold; wherein the second mold comprises a compartment into which said first template mold can be placed;
mounting the first template mold into the compartment of the second mold, thereby obtaining an operative mold;
casting a fluid casting material into said operative mold to obtain after solidification of said casting material an infiltrated template mold comprising a solid article that is at least partially located within the first template mold; and
separating said solid article from the first template mold by dissolving the sintered ceramic article of the first template mold with a suitable solvent, for example water.

In an advantageous variant of such a manufacturing method according to the invention, the solid article and the first template mold are removed from the second mold before dissolving the sintered ceramic article of the first template mold.

In another advantageous variant of such a manufacturing method according to the invention, the solid article is positively locked in the first template mold.

In a further advantageous variant of such a manufacturing method according to the invention, the second mold is a permanent mold or a lost mold.

In yet another variant of such a manufacturing method according to the invention, a metallic material or a polymer material or a ceramic material or a composite material is used as the casting material.

Another aspect of the invention concerns cast solid articles, manufactured with a method according to the invention as discussed above.

Such a cast article can be a tissue scaffold and/or a medical implant and/or a medical device.

### BRIEF DESCRIPTION OF THE DRAWINGS

In order to facilitate a fuller understanding of the present invention, reference is now made to the appended drawings and figures. These references should not be construed as limiting the present invention and are intended to be exemplary only.

Components that are identical, or that are identical at least in terms of their function, are designated below by identical or similar reference numbers.

FIG. 1 shows the results of rheological measurements on various printing resins: (a) Amplitude sweeps in oscillation of printing resins with an increasing NaCl concentration; (b) apparent yield stresses were extracted from (a) and plotted against the concentration of NaCl; (c) oscillatory rheological results of printing resins containing increasing amounts of a dispersant AOT; (d) apparent yield stresses extracted from (c) plotted against the dispersant concentration; (e) rheological data of the stress-controlled steady state measurement of printing resins with varying amounts of a diluent camphor; (f) viscosity values extracted from measurement (e) at a shear strain of 30 s⁻¹.

FIG. 1A provides an overview of the manufacturing workflow. Schematics depicting the layer-by-layer stereolithographic printing of a resin containing NaCl particles, surfactant, photocurable monomers, UV blocker, photoinitiator and a non-reactive diluent. The printed body is calcined to remove the organics and further sintered to obtain a dense, binderless NaCl body. This mold is infiltrated by a desired material and finally leached to obtain the positive complex-shaped body.

FIG. 1B shows the rheological characterization of salt-containing inks. The influence of the (a) surfactant, (b) salt and (c) diluent concentrations on the yield stress (τ_{y}) and apparent viscosity (η) of the inks. (a) 65 wt % NaCl, 1.75 wt % AOT, surfactant concentration varied; (b) NaCl concentration varied, 1.75 wt % AOT, 30 wt % diluent; (c) 65 wt % NaCl, AOT concentration varied, 30 wt % diluent. No yielding is observed for inks with surfactant concentrations below 0.1 wt % (with respect to (wrt) NaCl), which makes them unsuitable for our printing process. Data points with a cross (x) indicates the optimal composition that was identified.

FIG. 2 shows the results of exposure tests for different photoinitiator and UV blocker concentrations. (a) The layer thickness zₚ after exposure is plotted against the applied energy dose D during the exposure, for different concentrations of photoinitiator. (b) The critical energy dose D_{c} determined from the extrapolated fits in (a) are plotted against the photoinitiator concentration. (c) The layer thickness zₚ after exposure is plotted against the applied energy dose D during the exposure, for different concentrations of UV blocker. (d) The penetration depth hₐ determined from the extrapolated fits in (c) are plotted against the UV blocker concentration.

FIG. 2A depicts the photo-polymerization behavior and printing fidelity of salt-containing resins. (a,b) The influence of the applied light dose (Dₘₐₓ) on the cure depth (zₚ) of inks containing initiator concentrations in the range 1-5 wt % with respect to monomer. Fixed UV blocker contents of (a-b) 0 wt % and (c-d) 0.05 wt % were used in these experiments. The corresponding light penetration depth (hₐ) and critical dose (D_{c}) obtained by fitting the modified Lambert-Beer law to the experimental data are indicated in (b) 0 wt % and (d) 0.05 wt % of UV blocker.

FIG. 2B highlights the printing fidelity of inks with 0 wt %, 0.05 wt % and 0.125 wt % UV blocker for (a) negative and (b) positive features printed at a cure depth (hₐ) of 80 μm. The insets depict the average standard deviation (Std.) of all size measurements (0-2 mm) for different UV blocker concentrations. Optical microscopy images show representative examples of the 2 mm designed features, whereas the dashed lines indicate the theoretical size thereof.

FIG. 3 shows originally identical sample objects subject to different post-printing washing routines according to Table 5, the printing resin used to print these samples containing only HDODA as monomer: (a) images of green bodies, (b) images of sintered bodies. (c) depicts similar sample objects after sintering, using a printing resin containing 85 wt % IBA and 15 wt % TMPTA as monomers. Top row: side view, bottom row: top view.

FIG. 4 shows photographs of exemplary sintered NaCl molds manufactured according to the invention, including closed and open molds and gyroid shaped templates.

FIG. 4A depicts (c) the measured cracked area in printed cubes of different side lengths after heat treatment at 200° C. (A and C) or 690° C. (B and D) for 0 wt % camphor (A and B) and 30 wt % camphor (C and D). (a,b) Optical microscopy images in light transmission mode illustrate the effect of 30 wt % camphor on the formation of cracks (bright areas). Scale bars: 1 mm. The labels I-IV correspond to the experimental conditions detailed in (c).

FIG. 4B shows (a) the shape changes upon drying of model layers printed using salt-containing resins with or without camphor. All model layers were illuminated on the bottom side, and a salt-free resin is used as control. (b) Proposed mechanism for crack inhibition in inks containing camphor and salt particles. (c) The figures indicate the distinct cross-linking densities and amount of residual monomer expected at the bottom (B,D) and top (A,C) of a single printed layer. (c) Schematics showing the presence of a percolating network of salt particles resulting from the shrinkage of the polymer continuous phase upon heating of the printed material to 200° C. Shrinkage of the polymer phase (dotted) beyond particle jamming results in detachment from the particles and the formation of interstitial pores, as indicated in (d). (d) SEM image, Scale bar: 2 μm.

FIG. 4C illustrates the pore analysis and mechanical analysis of printed parts. (a) Pore size distribution and total surface area (S. Area, BET Analysis, Inset) of printed inks without (filled) or with (empty) camphor after drying at 30° C. and 200° C. (b) Storage (G′) and loss (G″) moduli of printed bars subjected to dynamic mechanical analysis in torsion mode. Samples were heated to 230° C., followed by an isothermal hold at 230° C. for 20 min.

FIG. 4D shows the results of the nitrogen gas sorption analysis of resin samples without NaCl. Pore volume distribution (a, DFT analysis) and total surface area (b, surface area, BET analysis) of casted resins without (filled) or with (empty) camphor after drying at either 30° C. or 200° C.

FIG. 5 shows (a) the results of thermogravimetric analysis of a printed part showing the mass loss as a function of temperature, and (b) the results of dilatometry for different sintering temperature, with the differential change in length and the temperature displayed as left and right y axis, respectively, as a function of time.

FIG. 5A illustrates the thermal analysis of the debinding process of printed NaCl-based cylindrical samples. (a) Thermogravimetric analysis (TGA) of samples containing no camphor (0 wt %), 30 wt % camphor (30 wt %, 8.5 s) and samples printed with an increased illumination time per layer (30 wt %, 15 s). (b) Corresponding results of the differential scanning calorimetry (DSC) analysis. Endothermic reactions are pointing upwards. The baselines are indicated in bright gray.

FIG. 6 shows exemplary debinding and sintering cycles, (a) with debinding (solid line) and sintering (dashed line) performed in separate cycles, and (b) with a combined debinding and sintering cycle.

FIG. 7 shows the influence of sintering parameters on relative density and shrinkage. (a) and (b) show the relative density and the shrinkage as a function of AOT concentration. (c) and (d) display the relative density and the shrinkage in z-direction as a function of the sintering temperature. (e) and (f) depict the relative density and the shrinkage as a function of the sintering time.

FIG. 8 shows SEM images illustrating the influence of the sintering parameters on the microstructure of a sintered NaCl mold, namely the influence on the density and surface quality of the sintering temperature and sintering time for different AOT concentrations. (a) 0.1 wt % AOT, tₛ=0 h, Tₛ=650° C., (b) 0.1 wt % AOT, tₛ=8 h, Tₛ=650° C., (c) 5 wt % AOT, tₛ=0 h, Tₛ=650° C., (d) 0.1 wt % AOT, tₛ=0 h, Tₛ=730° C.

FIG. 9 shows a sintered, printed NaCl structure (left) with SEM images of its microstructure with increasing magnification (two middle images) and EDS measurements (right) for Na, S, O and Cl.

FIG. 10 shows the influence of the dispersant AOT and the sintering aid Na₂SO₄ on the microstructure of sintered NaCl bodies. (a) 1.75 wt % AOT (with respect to NaCl), 0 wt % Na₂SO₄; (b) 0 wt % AOT, 1.75 wt % Na₂SO₄; (c) 0 wt % AOT, 0 wt % Na₂SO₄.

FIG. 11 schematically shows exemplary methods for the infiltration process: (a) casting in a beaker and vacuum assisted infiltration in a desiccator; (b) casting at elevated temperature in a melting set-up, where the NaCl mold is placed in an outer mold and casting is performed by application of an external overpressure; and (c) injection molding; while (d) schematically shows the removal of the template mold.

FIG. 12 highlights complex-shaped structures made through infiltration and leaching of salt molds. Comparison of (1) gyroid digital model (.stl model) with (2) micro-computed tomography (microCT) analysis of a printed, (3) sintered salt template, and (4) molded and leached silicone scaffold. X and Y denote specific cross sections, as indicated in (1). The linear shrinkage along different directions is indicated (next to the dashed box for comparison) as a percentage of the initial digital model. The scale bar of 5 mm is valid for (1-4).

FIG. 13 depicts the cell viability analysis of gyroid scaffolds prepared by leaching the 3D printed NaCl templates from infiltrated samples (either silicone or PCL). Top: Quantitative analysis of cell viability from live/dead assay on three different scaffold types made from silicone or PCL (n=5-10). The cell viability was >94% 2 days after seeding for all three scaffolds. Bottom: Representative confocal laser scanning microscopy image showing live/dead assay results of MC3T3-E1 pre-osteoblasts that have been seeded on a fibronectin-coated silicone scaffold with a pore size of 150 μm.

FIG. 14 shows examples of sintered salt molds and the corresponding complex-shaped structures of a range of materials obtained after infiltration and leaching steps. Top left: tracheal stent made of bioresorbable poly(DLLA-co-CL) copolymer. Top right: ultralightweight octagon lattice made from an Al—Si 12.6% metallic alloy. Bottom left: edible bunny made from dark chocolate. Bottom right: hollow reinforced tube made by covering a salt core with carbon fiber composite.

FIG. 15 illustrates complex-shaped structures obtained by casting or infiltration of various materials of interest into salt templates and leaching thereof. (a) Hierarchically porous PCL prepared by combining conventional salt leaching with microparticles (small porosity) with the templating approach presented here. (b) Casted, porous magnesium with a gyroid pore structure; (c) Injection-molded polystyrene scaffold with highly controlled pore structure.

FIG. 16 shows photographs of a sintered NaCl mold manufactured according to the invention (left) and a cast epoxy part made with said mold, after infiltration and dissolution of the NaCl mold (right).

### DETAILED DESCRIPTION OF THE INVENTION

A process to prepare a salt-based slurry for photopolymerization is described.

The obtained slurry is gelled by radiation curing, particularly in a DLP stereolithography printing step, and a green body is obtained. Non-gelled slurry is further removed, such as by rinsing and/or sonication in a solvent with low salt solubility. The cleaned article is further heat treated, so that the polymer matrix of the green body is pyrolyzed, and a binderless object consisting essentially of inorganic salt particles is obtained. The binderless article is then sintered to obtain a mechanically stable mold with a dense surface. During sintering potential residuals of the binder are removed. The sintered mold is used as a template for infiltration with a fluid material such as a powder, liquid or molten material, which can be further solidified by temperature, light or chemical reaction. Alternatively, the sintered article can also be used as a core for dip coating, spray coating or wrapping. Finally, the sintered mold is dissolved by an appropriate polar solvent, preferably an aqueous solution, and the final cast product is obtained.

### Materials Used

The following compounds have been used for the tests and experiments described in this description:
Camphor ((±)1,7,7-trimethylbicyclo[2.2.1]heptan-2-one, CAS No. 76-22-2, Alfa Aesar)
Sodium bis(2-ethylhexyl) sulfosuccinate, also commonly called sodium dioctyl sulfosuccinate (AOT) (CAS No. 577-11-7, 98%, Sigma Aldrich)
Ethanol (absolute for analysis, Merck KGaA, Germany)
Ethylene glycol (for analysis, Merck KGaA, Germany)
Phenyl-bis-(2,4,6-trimethylbenzoyl)-phosphinoxide (BAPO) (CAS No. 162881-26-7, Irgacure 819, BASF)
Isopropanol (99.8%, Sigma Aldrich)
Sodium chloride, NaCl (CAS No. 7647-14-5, Sigma Aldrich)
Olive oil
1-Phenylazo-2-naphtol (Sudan I, CAS No. 842-07-9, Sigma Aldrich)
1,1,1-Trimethylolpropane triacrylate (TMPTA) (CAS No. 15625-89-5, 98.5%, abcr GmbH)
1,6-Bis(acryloyloxy)hexane (HDODA) (CAS No. 13048-33-4, >85% GC, Tokyo Chemical Industry Co., Ltd.)
Isobornyl acrylate (IBA, CAS No. 5888-33-5, 93%, abcr GmbH)

### Additive Manufacturing

Additive manufacturing (AM, also known under the synonymous term 3D printing) is a manufacturing technique where an object is built up layer by layer in an additive manner. It enables the production of highly complex structures, which cannot always be achieved with conventional manufacturing techniques. Various additive manufacturing techniques have been developed over the past years, such as direct ink writing (DIW), selective laser melting (SLM), and stereolithography (SL). Stereolithography is the method that is advantageously applied for the given invention.

To produce an object with additive manufacturing, a three-dimensional model of the object, for example in the form of computer-aided design (CAD) file, is sliced into a series of 2D cross-sectional layers before it can be printed. In stereolithography, each of these thin layers of photocurable resin will be cured consecutively on top of each other. The resin usually consists of photosensitive monomers such as acrylates or epoxides, which will polymerize upon illumination [27, 28].

Stereolithography can be divided into two classes, with the main difference being how the actinic radiation (typically UV light) is applied. Either selected parts of the whole layer are cured at once using a light mask, or a laser beam scans over the surface, only polymerizing small volumes, so called strands, one by one. These two methods are called projection-based stereolithography (PSL), also known as direct light processing (DLP), and scanning-based stereolithography (SSL), respectively. SSL cannot reach a resolution as high as PSL because the scanning laser beam has an under width limit. Moreover, PSL is less time consuming because each layer is solidified in one step. However, SSL has higher radiation intensities and is advantageous for larger objects, although some of the resolution is lost.

A stereolithography apparatus (SLA), generally comprises five parts: resin tank, recoater, print platform, optics, and control systems. The recoater is mainly needed for high viscosity resins because they do not flow back and level in the tank. The printing platform is coupled to an elevator which controls the up- and downward movements of the platform while printing. The light source is usually a laser (Diode, He—Cd, Ar) coupled to an acoustic optical modulator to quickly turn on and off the laser. For direct light processing, usually a LED-LCD system is used as the light source.

In this description, a stereolithography apparatus will alternatively be named as stereolithography printer or 3D printer.

The photopolymerizable resin used for stereolithography will alternatively be named as photopolymerizable slurry, photocurable resin, printing resin, or 3D printing ink.

There are two possible setups for a stereolithography printer, namely top-down or bottom-up.

For the top-down orientation, the light source is placed on top, and the printing platform is inserted in the tank just below the resin surface. After each exposure, the platform moves downward by one layer height. The resulting objects are thus printed in upward direction. This approach has some disadvantages such as the need for large amount of resin and the extensive time for the resin to equilibrate again after the stage moved downward. Further, it is very challenging to control the layer thickness because only gravitational forces act on the fresh layer of resin on top of the already cured one. The use of the recoater blade is crucial in mitigating this problem as well as the use of a rather low-viscosity resin.

In contrast, the bottom-up approach circumvents most of these issues. The light source is placed on the bottom and the print head is placed on top. The printing platform is moved to the bottom of the resin bath where a thin layer of resin is polymerized on the platform surface through a transparent, anti-adhesive window in the bottom of the tank. After illumination, the tank is tilted in order to detach the cured layer from the tank and to allow the resin to flow back and form a fresh layer on the tank bottom. This allows for the use of considerably less resin compared to the top-down approach. After each layer, the print head is moved upwards by one layer height to cure the next layer. The resulting objects are hanging from the print head upside down. Another advantage compared to the top-down setup is the avoidance of a height limit of the object due to the tank size. The layer thickness can be controlled better, because it depends on the platform elevator and not on the fluid properties of the resin, which leads to higher vertical resolution as well as better surface quality of the object. However, the major drawback of the bottom-up approach is the requirement for an anti-adhesive and transparent bottom surface of the tank. Coatings such as Teflon or silicone are needed to easily detach the cured resin from the bottom, while it continues to adhere to the print platform.

In order to print complex geometries with high spatial resolution, it is crucial to understand and analyze the influence of the fundamental parameters in stereolithographic printing that determine the width and depth of the exposed layer and how they depend on the applied energy dose. There are two groups of parameters, a first group related to the printing process such as the laser power, layer thickness and hatch spacing, and a second group determined by the resin, including penetration depth and critical energy dose.

During printing, the photoinitiator turns into a radical upon irradiation with a suitable light source and hence initiates a free radical polymerization reaction of the monomer. This reaction continues as long as the resin is illuminated and will lead to a soft solid once the gelation point of the resin is reached. The intensity of the light decreases in z direction and xy direction of the resin away from the focal point on the surface. The gelation point can only be reached if the energy dose at that depth D(z) is above a critical energy dose (D_{c}). The polymerized thickness zₚ, also called cure depth, is the depth z at which the energy dose is just sufficiently high that the resin composition reaches the gelation point. The absorption of the energy of the light beam in the resin follows a modified version of the Beer-Lambert law:

zₚ=hₐ ln(D/D_{c}), (1)

where D is the applied energy dose, zₚ is the resulting cure depth, hₐ is the penetration depth (the cure depth when D=D_{c}), and D_{c} the critical energy dose.

The parameters D_{c} and hₐ depend on the resin composition and the spectrum of the applied actinic radiation and should be determined before printing, to assess the optimal printing parameters for a certain composition. Similarly, photoinitiator and UV blocker concentrations can be optimized for a given energy dose and a desired layer thickness.

The use of a UV blocker in a photocurable resin allows to reduce the penetration depth hₐ and xy resolution for a given critical energy dose D_{c}, since part of the actinic radiation is absorbed by the UV blocker when passing the resin. The decreased penetration depth hₐ leads to a decreased production speed, since either the illumination time has to be increased to reach the same polymerized thickness or more layers have to be produced. However, the decreased penetration depth also reduces the sensitivity of the cure depth towards slight changes of the light intensity. Such changes can occur for example due to clouding of the non-adhesive film of the resin tray or due to slight variations in the illumination time, changing illumination intensity and time, respectively. Therefore, an intermediate UV blocker concentration is often desired in the resin to accelerate the printing process without making the resin too sensitivity to unavoidable variations in illumination intensity.

The monomer conversion rate depends on the depth z (distance from illuminated surface), as predicted from equation (1). The degree of polymerization is largest at the surface and decreases exponentially thereafter. This irregularity within each layer can cause problems in further processing steps due to shrinkage during polymerization. As a result, mechanical stress is present and deformations appear, which may lead to crack formation of the objects. This defect is known as print-through. Furthermore, there needs to be some overcure, meaning that each strand should slightly penetrate the adjacent layers in order to avoid layer delamination, i.e. the separation of adjacent printed layers. It is advantageous to overcure 10%-35% in z height to avoid common printing defects such as delamination or print-through.

For a transparent resin, a cured line of the resin has the same shape as the intensity distribution of the light source, which corresponds to a Gaussian curve. However, if the resin contains inert particles, as in the photopolymerizable slurries according to this invention, the profile of the cured line is altered significantly due to scattering of the light beam. This leads to a broadening of the beam, especially for large differences in the refractive index of the particles and the monomeric phase [27,29]. Hence, a lower resolution is obtained. This effect can be mitigated for example by matching the refractive indices of the particles and the monomer, reducing the solid loading and adjusting the size distribution of the particles.

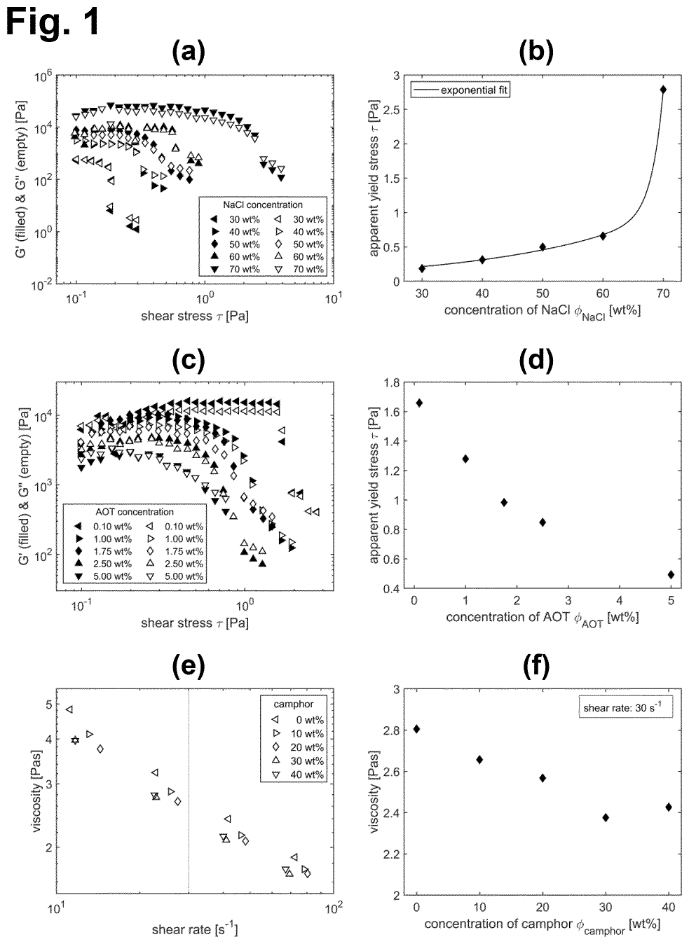

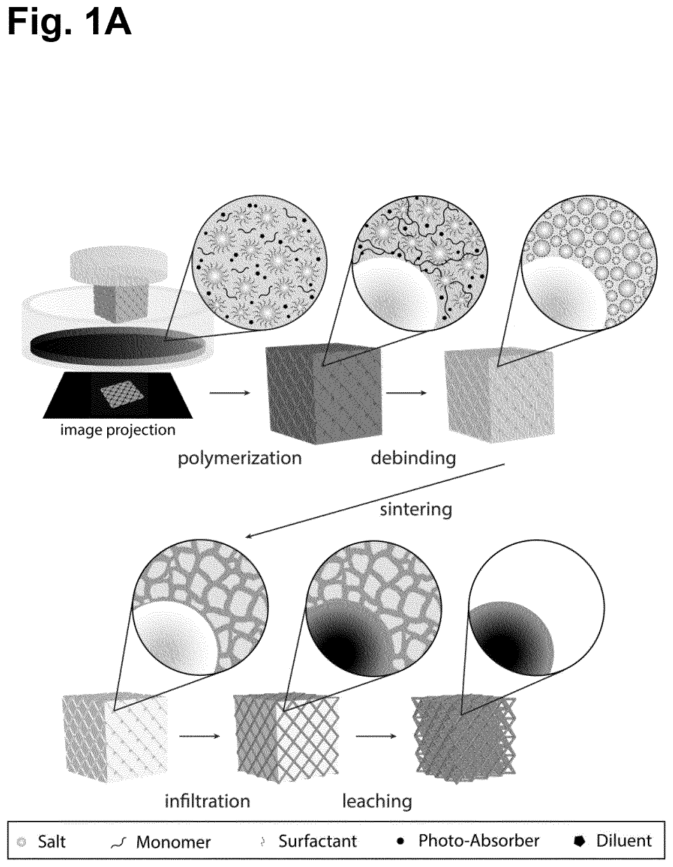

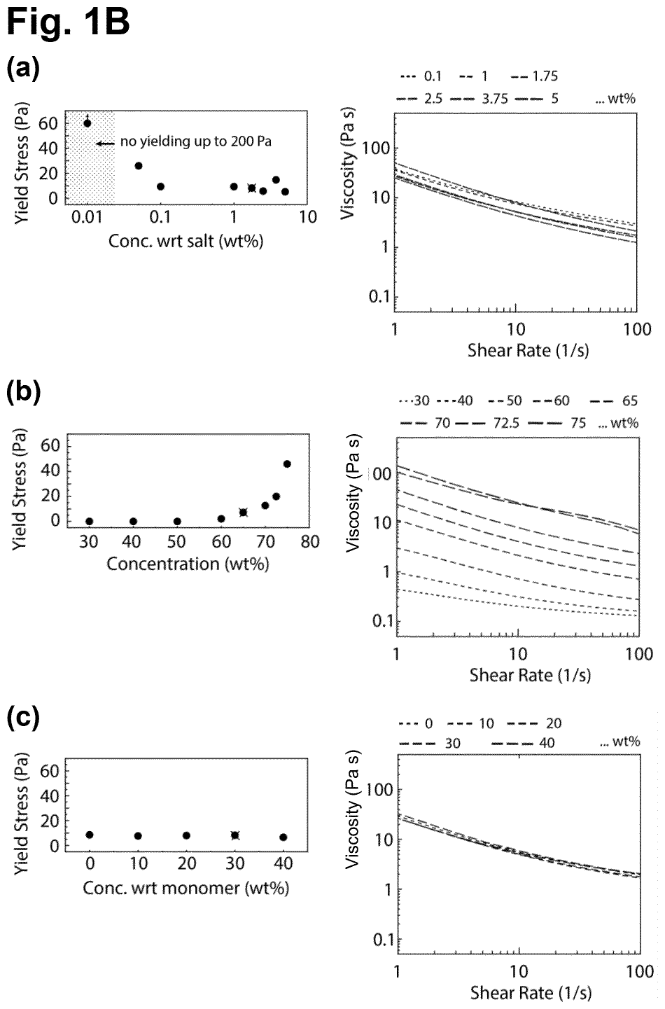

Method: The additive manufacturing of complex-shaped objects using salt templates involves a series of processing steps that includes light-based printing (i.e. vat photopolymerization), debinding, sintering, infiltration and leaching processes (FIG. 1A). The process is designed to generate a negative mold of dense salt that can be infiltrated with the material of interest and afterwards leached in water to create the desired complex-shaped object. Stereolithographic printing of the salt mold is achieved by suspending salt particles in a photo-curable resin. After fulfilling its shaping function during printing, the cured resin is thermally removed to provide a salt green body that is sintered into a dense negative mold at 690° C. To facilitate the removal of the polymerized binder, a diluent phase is often incorporated in the resin formulation. Diluents are removed at an earlier stage of the debinding process and hence decrease the risk of cracking during removal of the polymerized phase. Due to the thermal stability of the salt, the negative mold can be infiltrated by liquified or molten materials at temperatures up to ˜720° C., at which temperature the material starts to soften. The final positive object is obtained upon cooling of the infiltrated template followed by simple dissolution of the salt phase with water at room temperature.

In the context of the invention, 3D printing of the structures was carried out with a commercial bottom-up direct light processing (DLP) device (Original Prusa SL1, Prusa Research a.s.). The objects were designed in Solidworks (Solidworks 2020-2021, Dassault Systemes SolidWorks Corporation) and further edited in the original slicer software of the 3D printer (PrusaSlicer Version 2.2.0, Prusa Research a.s.).

Before printing, the printing resin was always vortexed until it was well homogenized (vortex-genie 2, Scientific Industries, Inc.). Alternatively, the printing resin can be homogenized in a mixer (Thinky Mixer ARE-250, Thinky cooperation).

The 3D printer was calibrated prior to every use and the illumination time was set to 40 s for the first four layers and to 22.5 s for the consecutive layers (for resins with 0.125 wt % Sudan I and 3 wt % BAPO. Alternatively, a minimally required exposure time to reach 80 μm cure depth was calculated from the resulting light penetration depth (hₐ) and critical energy dose (D_{c}) for each ink individually. In order to increase the adhesion at the printhead, the initial layers were exposed for 30-50 s with 10 fading layers to reach the desired illumination time. The layer thickness was set to 50 μm.

The required illumination time heavily depends on the slurry composition such as type, concentration and reactivity of monomer, initiator and inhibitor. The illumination time can be calculated from the working curve measured from an exposure test of the slurry according to a pre-established protocol [30].

The printing was performed at room temperature. However, slight heating may be applied to further decrease the viscosity facilitating the printing process.

### Particle Preparation

Particles of commercially inorganic salts, such as for example regular NaCl particles, are generally not suitable to be directly incorporated into a 3D printing ink, because the particle size exceeds the printing layer height, which is usually between 25 μm and 100 μm. Therefore, the particle size needs to be decreased.

In ceramic stereolithography (CSL), it is common to use particles with a size range of 0.05 μm to 10 μm. Such particle size ranges can be readily obtained by planetary ball milling. The advantage of ball milling over a direct synthesis of particles with the required size is the high efficiency of ball milling, and the limited possibilities for synthesizing salt particles with a certain, small particle size.

Typically, ceramic milling balls are used to produce enough kinetic energy to break the particles into smaller ones. The size of the milling balls has a large influence on the resulting particle size and particle size distribution. Finding the optimum diameter of milling balls is a compromise between the kinetic energy available for breaking the particles and the number of contact points of milling balls where particles can be broken. For a given weight of milling balls, the number of milling balls n increases with decreasing ball diameter d, with the relation n∂d⁻³. The number of contact points between the milling balls can be understood as the coordination number N arising from studies about dense packing of spheres. Since the coordination number is independent of the sphere diameter, the number of milling balls for a fixed weight of milling balls can be assumed to be proportional n∝N. This results in the combined relation n∝N∝d⁻³. The kinetic energy depends on the mass of the milling balls as well as the rotation speed of the ball mill. It is advantageous to use a mix of various milling ball sizes, in order to get μm sized particles with a narrow particle size distribution. Nevertheless, all the milling parameters have to be considered to find the optimal ball size variation for the desired particle size and particle size distribution. For the use of such particles in a resin, a narrow size distribution is favorable for high spatial resolution and a fine and smooth surface of the ceramic in the end.

In order to obtain a stable suspension and keep the particles from sedimenting and agglomerating, a surfactant or dispersant, respectively, can be used to modify the surface of the particles and to stabilize them in the liquid components of a photopolymerizable slurry. The surfactant can either be added after a (dry) milling process, or before milling. The latter case is more advantageous, since it reduces one step in the process.

In the context of this description, the terms “surfactant” and “dispersant” will be used interchangeably.

In one advantageous approach for producing homogeneous, fine-grained NaCl particles for use in a polymerizable slurry that can be used as a printing resin for 3D printing, the NaCl particles are reduced in size and are functionalized with the surfactant dioctyl sulfosuccinate sodium salt (AOT). In a typical process, the NaCl and the AOT are dispersed in isopropanol and added to an alumina milling jar. The dispersion is wet milled with zirconia mixing balls in four different sizes for 2 hours at 200 rpm with 5 min milling and 5 min break (planetary ball mill PM100, Retsch GmbH). The diameters of the mixing balls are 9.7 mm, 7.6 mm, 4.9 mm, and 2.4 mm.

For a batch of 200 g NaCl, the total mass of the added mixing balls is 575 g. The obtained slurry of AOT-functionalized NaCl particles in isopropanol is dried overnight in the oven at 60° C. For the de-agglomeration of the dried, functionalized NaCl particles, the obtained powder is dry ball milled with half of the zirconia mixing balls used for the wet milling process (5 min, 200 rpm).

Alternatively, the NaCl particles may also be produced by precipitation or spray drying [31].

### Photopolymerizable Slurry

A salt particle loaded, photocurable resin used for stereolithography typically consists of salt particles, a continuous monomeric phase, a photoinitiator, a UV blocker, and a dispersant. The dispersant is used to prevent the particles from agglomerating and keeping them dispersed and stable in the resin. Additionally, a diluent can be added to decrease the viscosity for easier printing [13, 24, 32].

There are two main groups of photocurable resins, namely aqueous and non-aqueous resins. Currently, mostly non-aqueous systems are used owing to several advantages such as higher strength of the green body and a lower difference between the refractive indices Δn of the particles and the continuous liquid phase. Typically, the liquid phase mainly consists of epoxy or acrylate monomer as suitable polymer building blocks. For good printability of a particle-loaded resin with DLP stereolithography, it is important that the solid loading (i.e. the particle concentration) is sufficiently high while the viscosity of the resin is kept low. High solid loading is advantageous due to enhanced form and structure stability. High solid loading decreases crack formation, geometrical deformation, pore formation, and general shrinkage during sintering. Further, it is required to have a printing resin with a sufficiently small yield point, because the resin needs to flow back below the printing platform, in order to cure the subsequent layer. Additionally, a diluent can help to reduce the viscosity due to high solid loading. It is possible to add as much as 40 wt % diluent, this upper limit being given by the requirement to still have enough of the monomeric phase to ensure sufficient mechanical stability of the printed part and limited shrinkage.

A key requirement of the process is to produce a dense, crack-free salt template at the end of the sintering step. Cracking is a common challenge in ceramic printing, and may result from internal mechanical stresses developed during shrinkage of the printed object upon calcination and sintering. To reduce shrinkage and minimize cracking, the concentration of salt particles in the initial resin should be maximized without impairing the rheological properties needed for the stereolithographic printing process. More specifically, in terms of rheological behavior, the ink must be sufficiently fluid to replenish the print stage under the action of gravity. Previous studies have shown that this flowability is achieved if at a shear rate of 30 s⁻¹ the yield stress and apparent viscosity of the ink are kept below 10 Pa and 60 Pa·s, respectively [26, 59].

In FIG. 1(a), the loss modulus G″ and storage modulus G′ increase for increasing solid loading due to increasing internal forces and particle-particle interactions. Hence, higher shear stresses are required to overcome the interaction forces to make the printing resin flow. Upon stress application above the particle interaction forces, G′ drops below G″, meaning that the material changes its behavior from solid-like to liquid-like. The evolution of the apparent yield stress as a function of increasing solid loading is depicted in FIG. 1(b). To ensure a shear stress well above the flow point during printing, the shear stress by the resin's own weight was calculated. Assuming an area of 1 cm², a thickness of 1 mm, and a density of ρᵣₑₛᵢₙ=1.75 g/ml (calculated using the main constituents NaCl, IBA, TMPTA and camphor, see below Table 4), the stress σ needs to satisfy the following condition:

$$σ = \frac{F}{A} = \frac{{m}_{ink} \cdot g}{A} = \frac{{ρ}_{ink} \cdot {V}_{ink} \cdot g}{A} = \frac{1750 \cdot 0.1 \cdot {10}^{- 6} \cdot 9.81}{{10}^{- 4}} Pa = 17.2 Pa > {σ}_{flow} \qquad ( 2 )$$

where F the force acting on the surface area A. ρᵣₑₛᵢₙ, mᵣₑₛᵢₙ and Vᵣₑₛᵢₙ are the density, mass and volume of the resin, respectively, g is the gravitational acceleration and σ_{flow} is the flow stress. Technically, all flow points from the formulated resins are well below 17.2 Pa as depicted in FIG. 1(b). By fitting Equation (2) to the experimental data, a solid NaCl loading of 65 wt % was found to meet the threshold stress (σ_{flow}) for a printing resin.

To reduce crack formation, it is advantageous to use a monomer or a monomer mixture that exhibits a low polymerization shrinkage. The polymerization shrinkage for a variety of acrylate monomers is listed in Table 2 (cf. [34])

TABLE 2
Polymerization shrinkage of monomers HDODA, IBA, TMPTA

| | | experimental | calculated |
|---|---|---|---|
| monomer | No. of acryl groups | shrinkage [%] | shrinkage [%] |
| HDODA | 2 | 14.0 | 23.8 |
| IBA | 1 | 5.5 | 11.3 |
| TMPTA | 3 | 12.0 | 28.6 |

The photopolymerizable slurries are prepared by first dissolving diluent (camphor), UV blocker (Sudan I), and photoinitiator (BAPO) in the monomer composition through vigorous stirring, followed by the addition of the functionalized NaCl particles. The slurry is then thoroughly mixed two times, for 5 min at 800 rpm (Thinky Mixer ARE-250, Thinky cooperation). A zirconia ball (d=7.6 mm) was added to enhance the mixing process.

An exemplary composition of a photopolymerizable slurry is given in Table 3, with a possible range of alternative concentrations, and alternative compounds.

TABLE 3

| | | [% wt] | | |
|---|---|---|---|---|
| | | (possible | | Alternative |
| | Compound | range) | | compounds |
| Ceramic | NaCl | 65 | (40-85) | KCl |
| particles | | | | |
| Monomer(s) | IBA | 21 | (15-60) | HDODA, ESOA, |
| | TMPTA | 3.7 | | PPTTA, IDA, 2-HEA |
| Diluent | camphor | 10 | (0-40) | vegetable oils, octane, |
| | | | | decane, dodecane |
| Photoinitiator | BAPO | 0.5 | (0.001-2) | TPO |
| Inhibitor (UV | Sudan I | 0.03 | (0-1) | Tartrazine, Allura |
| blocker) | | | | Red AC |

ESOA: epoxidized soybean oil acrylate (CAS No. 91722-14-4); IDA: Isodecyl acrylate; PPTTA: Pentaerythritol-tetraacrylate (CAS No. 51728-26-8); TPO: Diphenyl(2,4,6-trimethylbenzoyl)phosphine oxide (CAS No. 75980-60-8)

Two advantageous variants of photopolymerizable slurries are discussed below. In a first variant a) 100 wt % HDODA monomer is used, while in a second variant b) a monomer mixture of 85 wt % TMPTA and 15 wt % IBOA is used.

Printing resin with both monomer compositions were first tested without diluent, a NaCl particle concentration of 60 wt %, and a photo initiator concentration of 1 wt % with respect to the monomer content.

The composition of three advantageous polymerizable slurries is given in Table 4.

TABLE 4
Composition of printing resin (polymerizable slurry)
Variant a)

| | compound | wt % (relative wt % monomers) |
|---|---|---|
| inorganic salt particles | NaCl | 64.50 |
| monomer composition | HDODA | 24.32 (100) |
| diluent | camphor | 10.42 |
| photoinitiator | BAPO | 0.73 |
| UV blocker | Sudan I | 0.03 |

Variant b)

| | compound | wt % (relative wt % monomers) |
|---|---|---|
| inorganic salt particles | NaCl | 64.50 |
| monomer composition | IBA | 20.67 (85) |
| | TMPTA | 3.65 (15) |
| diluent | camphor | 10.42 |
| photoinitiator | BAPO | 0.73 |
| UV blocker | Sudan I | 0.03 |

Variant c)

| | compound | wt % (relative wt % monomers) |
|---|---|---|
| inorganic salt particles | NaCl | 69.77 |
| monomer composition | IBA | 25.42 (85) |
| | TMPTA | 4.48 (15) |
| photoinitiator | BAPO | 0.3 |
| UV blocker | Sudan I | 0.03 |

In DLP stereolithography, the resin has to fulfill two main rheological requirements. First, it should have no or only a small yield point. Additionally, a low viscosity (below 5000 mPa·s) is advantageous [33]. While these two requirements are best met for low solid loadings, high loadings are beneficial for limiting the crack formation during pyrolysis and sintering and reduce risk of (anisotropic) shrinkage. Hence, a compromise between a high solid content and a low viscosity has to be found for maximizing the solid content in the printing resin, yet ensuring good printability.

The printing resin was characterized with rheological measurements in order to check its printability. During DLP printing, the resin is required to flow back below the print head to replenish the new layer. Rheological measurements of the resins containing varying concentrations of AOT or NaCl were carried out by stress-controlled oscillatory measurements on a rheometer (Anton Paar MCR 302, Anton Paar GmbH). A stainless-steel parallel plate with a diameter of 25 mm and a gap size of 1 mm was used. The plates were sand blasted to minimize wall slip. For very low viscous resins (low NaCl particle concentration), a gap size of 0.5 mm was chosen. All amplitude sweeps were carried out at 1 rad/s with a logarithmically increasing shear stress from 0.001 Pa to 100 Pa. The apparent yield stress σ_{flow} was determined from the crossover point from the storage modulus G′ and loss modulus G″ for each resin composition. For determining the viscosity of the resins with increasing diluent concentration, steady state measurements were conducted. The shear stress was logarithmically increased from 0.01 Pa to 1000 Pa. All of the rheological measurements were performed at 25° C. at a sample size n=1.

FIG. 1 shows the results of the rheological measurements on various printing resins, namely (a) amplitude sweeps in oscillation of resins with an increasing NaCl concentration; (b) apparent yield stresses were extracted from (a) and plotted against the concentration of NaCl; (c) oscillatory rheological results of resins containing increasing amounts of AOT; (d) apparent yield stresses extracted from (c) plotted against the surfactant concentration; (e) rheological data of the stress-controlled steady state measurement of resins with varying amounts of camphor; and (f) viscosity values extracted from measurement (e) at a shear strain of 30 s⁻¹.

Influence of solid loading: Oscillatory rheological data was acquired for NaCl particle concentrations varied between 30 wt % and 70 wt %. HDODA was used as photosensitive monomer in all compositions. FIG. 1(c) shows the evolution of the storage modulus (G′) and the loss modulus (G″) as a function of the shear stress. For all these resins, G′ dominates over G″ at low shear stress but drops below G″ at higher shear stresses. This intersection corresponds to the apparent yield stress, also called flow point. The flow point defines the stress at which the suspension changes from solid-like to fluid-like behavior. For an easy comparison between all resins, the apparent yield stress is plotted against the NaCl concentration in FIG. 1(b) and ranges between 0.2 Pa and 2.8 Pa. The higher the solid loading, the higher the apparent yield stress. Up to 60 wt %, the apparent yield stress increases only slightly. Between 60 wt % and 70 wt % NaCl, there is a significant increase from 0.7 Pa to 2.8 Pa. The data was fitted with an exponential function represented as a solid line:

$${σ}_{flow} ( φ ) = 1.552 \cdot {10}^{- 16} {e}^{0.5285 φ} + 0.06842 \cdot {e}^{0.038 φ} , \qquad ( 3 )$$

where σ_{flow} is the yield stress and p the solid loading of NaCl.

Influence of Surfactant on Flow Behavior: Because the NaCl particles are hydrophilic and have to be dispersed in the hydrophobic monomeric phase, the surfactant AOT is added to the printing resin. The surfactant modifies the surface of the particles to make them hydrophobic and hence decrease the particle-particle attractive interactions. In order to quantify the reduction of attractive interactions, the rheological behavior of the printing resin was investigated. Oscillatory amplitude sweeps were conducted to investigate the influence of the AOT concentration on the rheological properties. FIG. 1(c) shows the storage and loss modulus as a function of shear stress. All compositions undergo the transition from solid-like to liquid-like behavior at shear stresses higher than 0.1 Pa. Generally, lower AOT concentrations lead to higher apparent yield stresses. This trend is most prominent for low AOT concentrations.

FIG. 1(d) summarizes the apparent yield stresses from FIG. 1(c) and relates them to the surfactant. It was found that increasing AOT concentration, directly decreases the storage and loss modulus. Apart from the moduli, the apparent yield stresses clearly decrease with increasing AOT concentration as displayed in FIG. 1(d). In the presence of the surfactant, less shearing is necessary to overcome the internal forces holding the material together. For typical shear stresses of about 1 Pa present during DLP stereolithography printing, a concentration of 1.75 wt % AOT was determined as optimum. Although higher concentrations of AOT result in even lower apparent yield stresses, it is not beneficial for the system, since this can lead to bubble formation and defects of the printed parts.

Influence of Diluent on Flow Behavior: The composition of the printing resin is a compromise between high solid loading and low viscosity. Since the solid content strongly increases the viscosity of the printing resin, a diluent was added to counteract this effect. Camphor was used as diluent. An advantage of camphor is its melting temperature above room temperature and its quick dissolution in acrylates. Further, the printed parts gain additional strength after the solidification of camphor after the polymerization reaction. Data from steady state rheological measurements were acquired to investigate the influence of camphor as a diluent on the viscosity.

The concentration of camphor was altered from 0% to 40 wt % with respect to the total amount of liquid phase, as displayed in FIG. 1(e). The viscosity decreases with increasing shear rate; hence all compositions show shear thinning behavior. For an ideal Newtonian fluid, the viscosity is independent of the shear rate. During DLP printing, shear rates of approximately 30 s⁻¹ arise [33]. FIG. 1(f) depicts the viscosity as a function of camphor concentration at a shear rate of 30 s⁻¹. The viscosity varies between 2.4 Pa·s and 2.8 Pa·s and decreases with increasing camphor concentration, until a minimum is reached at a camphor concentration of approximately 30 wt %. For higher concentrations, the viscosity remains roughly constant. A concentration of 30 wt % camphor was identified as best because it resulted in the lowest viscosity and did not hinder the polymerization during the printing process. The use of camphor allowed the use of a high solid loading while keeping the viscosity sufficiently low.

A similar analysis was performed for a resin containing the monomers IBA and TMPTA, the diluent camphor as well as AOT functionalized NaCl particles. The rheological properties of inks with varying salt, surfactant and diluent concentrations were analyzed by viscosity measurements on a stress-controlled rheometer (Anton Paar MCR 302, Anton Paar GmbH). All measurements were performed at 25° C. with a parallel-plate setup using sand-blasted plates to minimize wall slip. The plates had a diameter of 25 mm and a gap size of 1 mm was employed. Steady-state measurements were performed by increasing the shear stress logarithmically from 0.01 to 200 Pa. The yield stress was defined as the point of sudden increase of shear stress in a double logarithmic shear strain versus shear stress representation of the data.

The rheological measurements revealed that the yield stress and the apparent viscosity, defined as the ratio between shear stress and shear rate, of the ink strongly depend on the surfactant (AOT) and salt concentration (FIG. 1B(a,b)). A minimum surfactant content of 0.1 wt % is needed to lower the attractive van der Waals forces between the salt particles and thus reduce the yield stress and the viscosity of the ink below the rheological limits set by the printing process. For inks containing 1.75 wt % AOT, a percolating particle network with well-defined yield stress and high apparent viscosity is starting to form for salt concentrations above 60 wt %. This imposes an upper limit of 65 wt % for the maximum salt content that can be incorporated in the resin. The partial replacement of the monomer mixture by a diluent at up to 40 wt % (with respect to monomers) does not affect the apparent viscosity and yield stress, suggesting that the rheological properties of the investigated ink are dominated by the interactions between the salt particles (FIG. 1B(c)). On the basis of this rheological analysis, a formulation with 65 wt % salt, 1.75 wt % surfactant with respect to NaCl, and 30 wt % of diluent with respect to monomer was chosen as a standard ink for the stereolithographic printing process as identified by the data points with a cross (x) in FIG. 1B(a,b,c).

The photoinitiator and UV blocker concentrations on the polymerization reaction have to be adjusted to maximize the resolution of the 3D printed parts, while avoiding print-through and delamination. While the initiator dominates the energy needed to initiate the polymerization, the UV blocker primarily affects the penetration depth.

To find the optimal photoinitiator and UV blocker concentrations for the photopolymerizable slurry, exposure tests have been carried out. Printing resins with a composition according to Table 4 have been used. Regular glass slides were covered with a piece of fluorinated ethylene propylene (FEP) film and further placed directly onto the liquid crystal display (LCD) of the 3D printer (Original Prusa SL1, Prusa Research a.s.). A sufficiently thick layer of printing resin was evenly spread on top of the FEP film, fully covering the glass slide. A rectangular area was split into 32 small rectangles which were exposed to near UV light (λ=405 nm) for different time periods, between 2 s and 64 s, with 2 s increments. After exposure, excess resin was removed, and the glass slides were rinsed with isopropanol and water. They were left to dry overnight before the thickness of each rectangle was measured with a micrometer (IP 54, Helios-Preisser GmbH).

Photoinitiator Concentration: BAPO was used as a photoinitiator. The photoinitiator concentration is relevant because the photoinitiator generates the free radicals for the polymerization reaction. If the initiator concentration is too low, the printing resin will not solidify or will not form a sufficiently deep layer, increasing risk of delamination or print-through. Contrarily, if there is too much initiator, overcuring takes place and reduces the resolution of the final part.

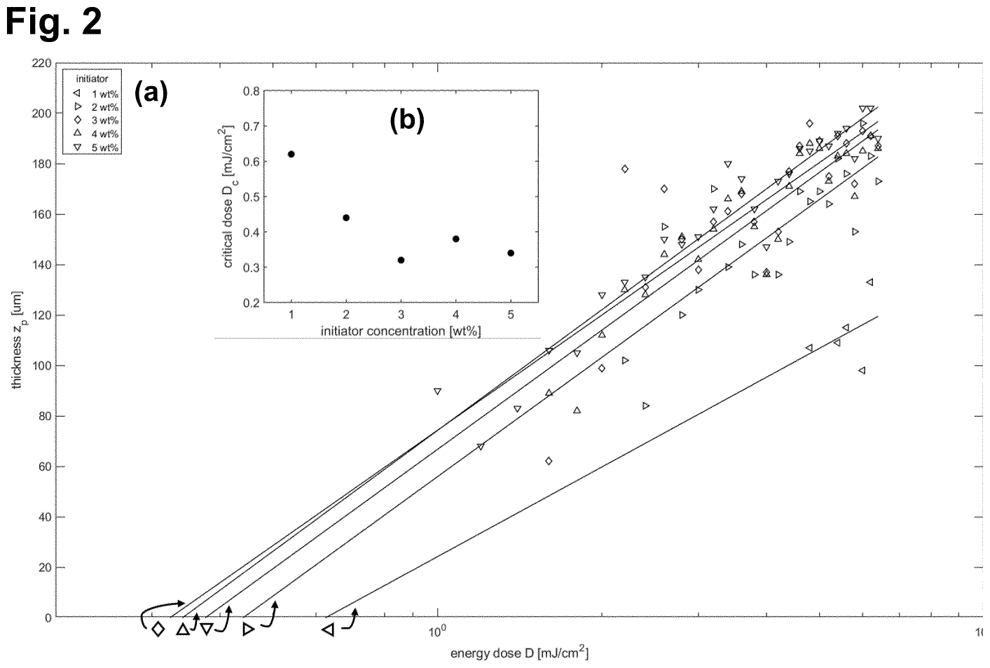

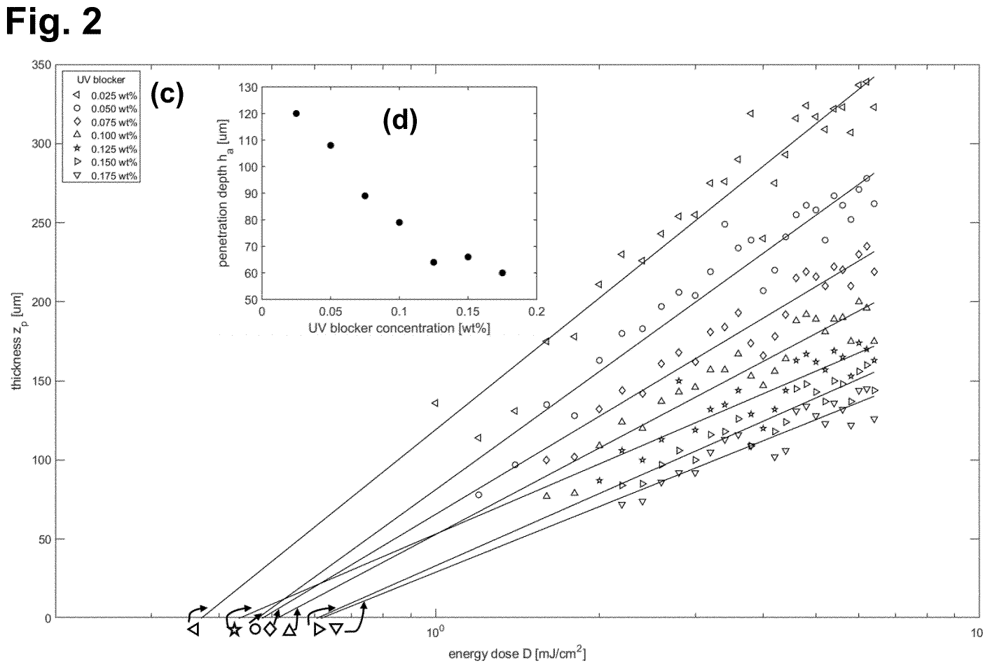

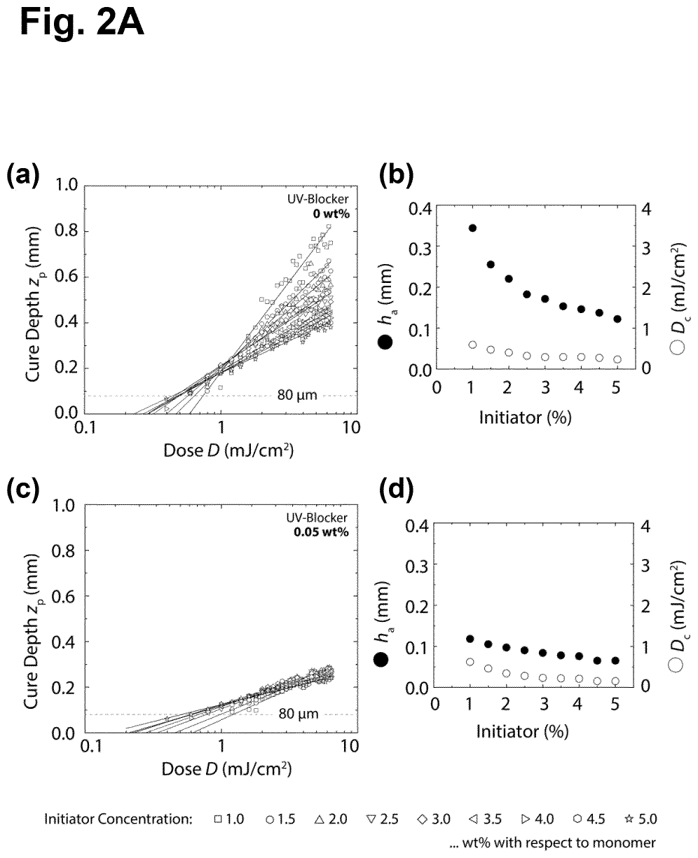

The photoinitiator (BAPO) concentration was varied from 1 wt % to 5 wt % with respect to the monomer concentration, while the UV blocker concentration was kept constant at 0.125 wt %. The layer thickness of each rectangle was determined after exposure according to the exposure method explained above. The energy dose was obtained by multiplying the exposure time with the light intensity of the light source (0.1 mW/cm²). FIG. 2(a) shows the polymerized layer thickness zₚ as a function of the applied energy dose D in a semi-Log plot. The measured thickness values range from 70 μm to 200 μm. The data points were linearly fitted, and the critical energy dose D_{c}, which corresponds to the intersection with the x-axis, was extrapolated. It should be noted that the slope corresponding to the penetration depth of the fit does not vary much with increasing photoinitiator concentration in the system (see Equation (1)). As displayed in FIG. 2(a), the thickness of the polymerized layer strongly increases when the BAPO concentration increases from 1 wt % to 2 wt %. Thereafter, it still increases with an increasing amount of initiator, but not significantly. This is due to more initiator molecules available resulting in a higher number of radicals, which initiate the polymerization reaction. Similarly, the thickness increases for higher radiation energy doses. An overall trend was detected and fitted with a line (FIG. 2(a)). Below D_{c}, no polymerization takes place.

FIG. 2(b) shows D_{c} as a function of photoinitiator concentration. The critical energy dose D_{c} varies significantly with changing photoinitiator concentrations. It ranges from 0.32 mJ/cm² for 3 wt % to 0.62 mJ/cm² for 1 wt %. First, D_{c} decreases with increasing initiator concentration. This is due to more reactive molecules that can initiate a polymerization reaction. Above 3 wt %, D_{c} levels off. Thus, increasing the photoinitiator initiator concentration above 3 wt % does not result in an earlier initiation of the polymerization reaction.

Likewise, exposure tests with 0 wt % and 0.05 wt % UV blocker and 1 wt % to 5 wt % photoinitiator (with respect to the monomer concentration) were performed (FIG. 2A). The effect of the initiator and UV blocker on hₐ and D_{c} are directly influenced by the concentrations of photon-absorbing and reactive species present in the ink. Taking the formulation without UV blocker (0 wt % Sudan I) as an example, it can be found that an increase in initiator concentration reduces hₐ substantially while keeping the D_{c} value nearly constant (FIG. 2A(b)). The drop in light penetration results from the fact that the initiator molecules are photon-absorbing species. The nearly constant D_{c} value suggests that the initiator concentration of 1 wt % is already sufficient to generate a high density of reactive monomer species for the polymerization process. Altered trends are observed when 0.125 wt % UV blocker is added to the resin (FIG. 2). In this case, the photo-absorbing molecules dominate the light penetration depth. The presence of a high UV blocker concentration in this resin decreases the relative fraction of activated photo-initiators in the mixture, which is translated into a much stronger effect of the initiator concentration on the critical dose.

In terms of critical dose, inks with low D_{c} are often desired to reduce the printing time. Since the printer operates at a fixed illumination intensity, a lower critical dose reduces the time the ink needs to be illuminated to generate the concentration of reactive species required for polymerization. With regards to the light penetration depth, formulations with low hₐ show a weaker dependence of the cure depth on the illumination dose. This makes the inks more stable against possible variations in the illumination dose resulting from manufacturing issues, potentially improving the reproducibility of the printing process.

UV Blocker Concentration: Due to the light scattering particles, the light penetration is in principle limited also in absence of a UV blocker. However, the very high penetration depth in such a system directly results in high sensitivity of the cure depth to slight process changes in illumination time or illumination intensity—both affecting the irradiation dose. Reducing the penetration depth for higher process stability will inevitably result in longer print duration. It is hence essential to find a good balance between stable and efficient printing for a desired resolution. Such a balance is usually found at a penetration depth of 2-4× targeted layer height, with a factor 2 favoring high resolution prints. For a layer thickness of 50 □m, a penetration depth of 100-200 □m is required. UV blockers must hence absorb light at the wavelength of the light used for printing. In the discussed 3D printing resins, Sudan I was used as a UV blocker.

For finding the optimal value and keeping the resolution high, exposure tests were conducted for different UV blocker (Sudan I) concentrations, in analogy to the exposure tests in FIGS. 2(a) and 2(b). The concentration of Sudan I was varied from 0.025 wt % to 0.175 wt % with respect to the total monomer concentration, while the Initiator concentration was kept constant at 3 wt %. FIG. 2(c) shows the polymerized layer thickness zₚ as a function of the applied energy dose D in a semi-Log plot. Lower UV blocker concentrations lead to a thicker polymerized layer for the same energy dose D because less light is absorbed by the UV blocker. These results show that the UV blocker is indeed effective. The thickness measured of the rectangles varies from about 80 μm to 350 μm for concentrations from 0.175 wt % to 0.025 wt % of Sudan I, respectively. The data points are linearly fitted for each concentration value. Looking at the two important parameters of Equation (1), namely the x-intercept D_{c} and the slope hₐ, the influence of the UV blocker concentration is larger on the penetration depth zₚ than on the critical energy dose D_{c}. FIG. 2(d) depicts the penetration depth hₐ as a function of the UV blocker concentration. It is clearly visible that hₐ significantly decreases from 120 μm to 64 μm for a concentration of 0.125 wt %. For higher concentration, the penetration depth remains roughly constant. Considering the best resolution and print speed balance, a penetration depth of 100 □m was targeted for a layer height of 50 □m. An optimal Sudan I concentration of 0.05 wt % was found to decrease the penetration depth of the slurry to 108 □m.

To evaluate the effect of the resin formulation on the fidelity and reproducibility of the SLA printing process, model parts with positive or negative features were printed and compared the experimentally obtained sizes with their nominal theoretical values (FIG. 2B). Experiments were performed with inks containing a fixed initiator concentration of 2 wt % and varying UV blocker contents of 0, 0.05 and 0.125 wt %. This allowed us to evaluate inks with different sets of D_{c} and hₐ values for their printing accuracy and reproducibility. By comparing experimental and nominal sizes, it was found that the fidelity of negative features is high for dimensions greater than 1 mm (FIG. 2B(a)). The opposite is true for positive features, which show sizes closer to their nominal values when smaller than 1 mm (FIG. 2B(b)). While the size accuracy was shown to depend on the exact composition of the ink, most formulations lead to very reproducible feature dimensions, as reflected in the low standard deviation values obtained. The exception to this trend is the ink prepared without light-absorber when used to print negative features. In this case, the strong dependence of the polymerization depth (zₚ) on the applied light dose (D) leads to poorly reproducible negative features across a broad size range (FIG. 2B(a)). To combine fast printing, high fidelity and high reproducibility, an ink formulation with 0.05 wt % UV blocker and 2 wt % photoinitiator that lead to low hₐ and D_{c} values was selected for the stereolithographic printing of the salt-laden parts

### Specific Examples of Photopolymerizable Slurries

Example 1: An intermediate slurry was prepared by ball milling a mixture consisting of 28.6 g HDODA, 69.6 g NaCl, and 1.8 g AOT. To prepare the slurry, 1.7 g HDODA, 0.024 g BAPO, and 0.00013 g Sudan I were added to 10 g of the intermediate slurry. The slurry was printed at a light intensity of 20 mW/cm², with an exposure time of 3 s and a layer thickness of 50 μm.

Example 2: Surface-modified NaCl particles were prepared by ball milling NaCl with 1.75 w/w AOT in isopropyl alcohol followed by drying at 60° C. overnight. A slurry was prepared by mixing 24.5 wt % HDODA, 10.5 wt % camphor, 3 wt % BAPO (with respect to monomer concentration), and surface-modified NaCl particles as balance. The critical energy dose and the penetration depth were determined from an exposure test as 0.26 mJ/cm² and 151 μm, respectively. Using a 3D printer with a light intensity of 0.1 mW/cm², an illumination time of 5 s was applied for each layer. The base layer illumination time was set to 15 s.

Example 3: Surface-modified NaCl particles were prepared as in example 2. A slurry was prepared by mixing 20.7 wt % IBA, 3.7 wt % TMPTA, 10.4 wt % camphor, 2 wt % Omnicure 819 (with respect to monomer concentration), 0.05 wt % Sudan I (with respect to monomer concentration), and surface-modified NaCl particles as balance. The critical energy dose and the penetration depth were determined from an exposure test resulting in 0.51 mJ/cm² and 119 μm, respectively. Using a 3D printer with a light intensity of 0.1 mW/cm², an illumination time of 10 s per layer was applied. The initial exposure time was 30 s.

Example 4: Surface-modified NaCl particles were prepared as in example 2. A slurry was prepared by mixing 20.0 wt % IBA, 6.7 wt % AESO, 8 wt % camphor, 3.9 wt % BAPO (with respect to monomer concentration), 0.125 wt % Sudan I (with respect to monomer concentration), and surface-modified NaCl particles as balance. The critical energy dose and the penetration depth were determined from an exposure test resulting in 0.18 mJ/cm² and 51 μm, respectively. Using a 3D printer with a light intensity of 0.1 mW/cm², an illumination time of 22.5 s per layer was applied. The initial exposure time was 30 s.

Example 5: Surface-modified KCl particles were prepared by ball milling KCl with 1.75 w/w AOT in isopropyl alcohol followed by drying at 60° C. overnight. A slurry was prepared by mixing 20.7 wt % IBA, 3.7 wt % TMPTA, 10.4 wt % camphor, 2 wt % Omnicure 819 (with respect to monomer concentration), 0.05 wt % Sudan I (with respect to monomer concentration), and KCl as balance. The critical energy dose and the penetration depth were determined from an exposure test resulting in 0.63 mJ/cm² and 197 μm, respectively. Using a 3D printer with a light intensity of 0.1 mW/cm², an illumination time of 9 s per layer was applied. The initial exposure time was 30 s.

Example 6: Sodium chloride (NaCl), bis(2-ethylhexyl) sulfosuccinate sodium salt (AOT) and 2-propanol (99.8% purity) were all purchased from Sigma Aldrich. To decrease the size of the as-received particles to an average particle size below 2 μm, 50 g of NaCl and 0.875 g AOT (1.75 wt % with respect to NaCl, except noted otherwise) were dispersed in 100 mL of 2-propanol (99.8%, Sigma-Aldrich). The dispersion was ball-milled in a planetary ball mill (PM100, Retsch GmbH) at 200 rpm for 2 h (including cooling breaks), at intervals of 5 min milling spaced by 5 min breaks. The milling was performed in a custom-made alumina jar using 575 g zirconia balls with diameters of 9.7 mm, 7.6 mm, 4.9 mm, and 2.4 mm. The milled slurry was dried at 60° C. overnight. To form the photopolymerizable resin, the non-reactive diluent camphor ((1R)-(+), 98% Alfa Aesar), the UV blocker Sudan I (Acros Organics) and the photoinitiator Omnirad 819 (iGM Resins) were fully dissolved in the monomers isobornyl acrylate (IBOA, technical grade, Sigma Aldrich) and 1,1,1-trimethylolpropane triacrylate (TMPTA, technical grade, Sigma Aldrich). The monomers were used at an IBOA-to-TMPTA weight ratio of 85:15. The concentration of camphor is always indicated as weight percentage of the liquid phase. UV blocker and -initiator were added at 0.05 wt % and 2 wt % with respect to monomer concentration, respectively (unless stated otherwise). To prepare the final ink, 65 wt % of milled NaCl particles were homogeneously dispersed in the photopolymerizable resin at 800 rpm for 10 min by using a planetary centrifugal mixer (Thinky Mixer ARE-250, Thinky Cooperation).

A minimally required exposure time to reach 80 μm cure depth was calculated from the resulting light penetration depth (hₐ) and critical energy dose (D_{c}) for each ink individually. In order to increase the adhesion at the printhead, the initial layers were exposed for 30-50 s with 10 fading layers to reach the desired illumination time. The layer thickness was fixed at 50 μm, unless stated otherwise. The print accuracy of negative and positive features was analyzed for various UV blocker concentrations to investigate its role on the attainable resolution. All inks were printed under exposure conditions to reach a targeted cure depth of 80 μm. Negative holes of varying diameter were printed in a 2 mm solid bar, parallel to the build plate. Positive bars of varying width and 1 mm height were printed on a baseplate. The samples were visualized with a Keyence microscope and the resulting images analyzed with ImageJ (ImageJ2, version 2.3.0/1.53f). Three measurements were conducted for each data point. Digital designs were oversized by 15% in the x- and y-direction, and 25% in the z-direction to compensate for the shrinkage due to the heat treatment. After printing, the samples were cleaned with 99.8% pure 2-propanol by 3×3 min sonication in an ultrasound bath. The samples were further dried at ambient conditions before characterization.

Washing after Printing

After 3D printing, excess printing resin with unreacted monomer has to be removed from the printed object, by washing the object in a solvent. Several combinations of various solvents for washing or sonication have been tested (see Table 5).

The test objects were hollow cylinders with cross-beams. After printing, the printed objects were immersed in a first solvent A, sonication was applied for 6 minutes, and the printed objects were subsequently rinsed with a second solvent B. Drying was conducted at ambient temperature and atmosphere. Moreover, the samples were dried with their openings up-side down for the remaining solvent to flow out by gravity.

TABLE 5
Washing routines

| Washing routine No. | Solvent A | Solvent B |
|---|---|---|
| 1 | isopropanol | isopropanol |
| 2 | 1^{st} isopropanol | water |
| | 2^{nd} water | |
| 3 | isopropanol | water |
| 4 | ethylene glycol | ethylene glycol |
| 5 | olive oil | olive oil |

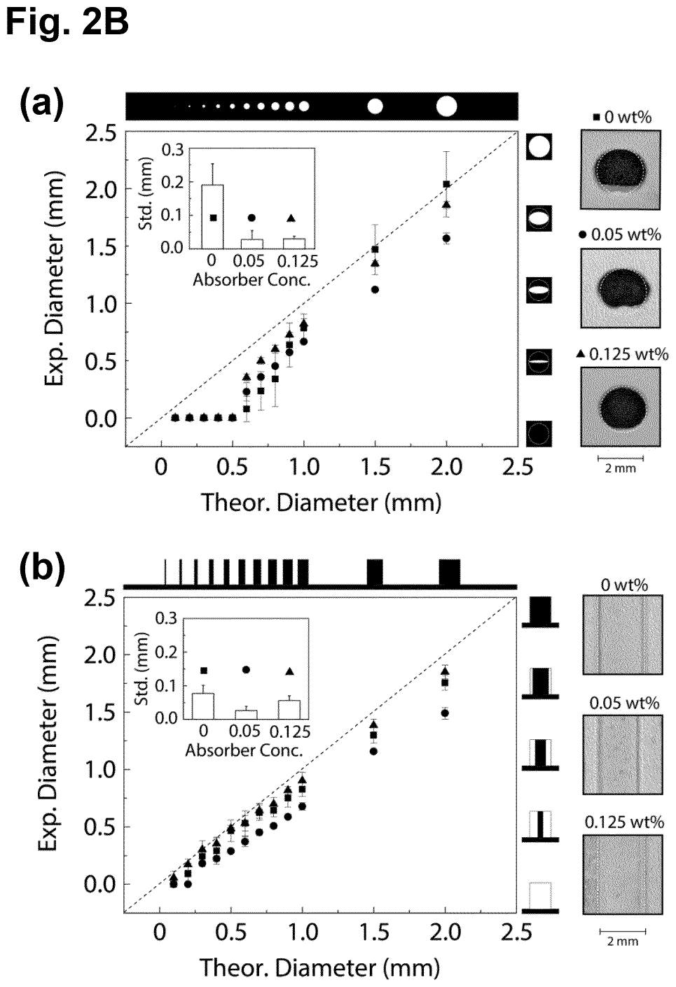

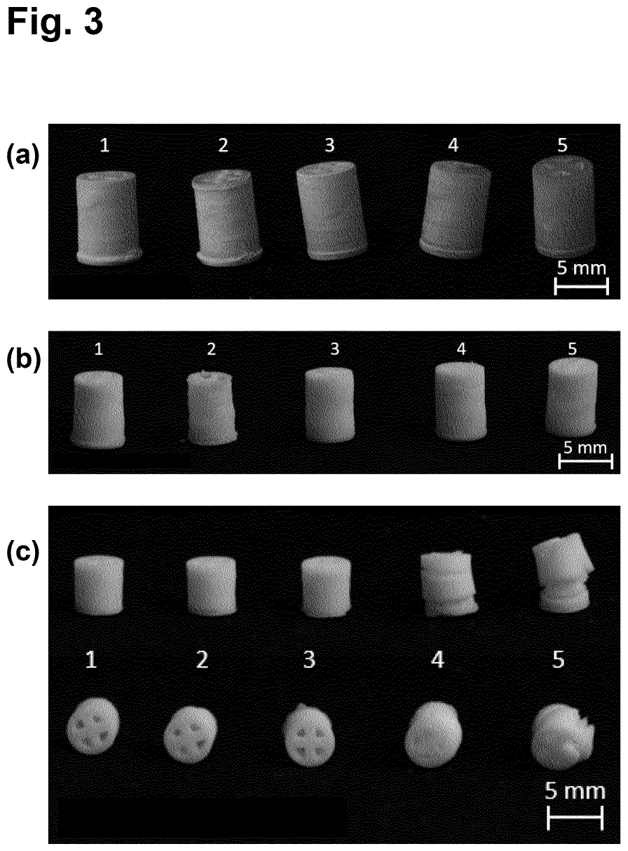

The results of the different washing routine tests and techniques were qualitatively analyzed based on the images displayed in FIG. 3. The samples shown in FIGS. 3(a),(b) were printed with a printing resin according to Table 4(a), whereas the samples in FIG. 3(c) were manufactured with a printing resin according to Table 4(b) and a solid loading of 70 wt %. The numbers ranging from 1 to 5 on each image in FIG. 3 correspond to the washing routine enumerated in Table 5.

It is clearly visible that the overall quality of the sintered samples is higher with the printing resin composition with IBA 85 wt %/TMPTA 15 wt %. It is assumed that this is the result of the more advantageous cross-linking effect of TMPTA in combination with IBA (which does not cross-link), compared to the cross-linking HDODA alone.

Visual inspection revealed that washing routine No. 2 is most detrimental, and leads to thinned out walls, and largely changed dimensions (see FIGS. 3(a),(b)). A lot of material (NaCl) was removed during washing. The walls are very thin, deformed and look porous. The thinning of the walls can also be detected for washing routine No. 2 in FIG. 3(c), but to a smaller extent. While not wishing to be bound to a specific explanation, it is postulated that sonication in water is detrimental to the green body structure, possibly to unwanted dissolution of salt particles close to the matrix surface.

The sample objects treated according to washing routines No. 4 and 5 had few small cracks and were not as stable as the sample objects treated according to washing routines No. 1 and 3.

Sample objects No. 4 and 5 in FIG. 3(c) exploded during sintering because there was still a lot of unreacted monomer in the cylinder, which was not completely washed out. One reason for this effect is that ethylene glycol is not miscible with IBA, and therefore cannot completely wash out the unreacted monomer. Since olive oil is well miscible with IBA (sample object No. 4, FIG. 3(c)), it is assumed that the unreacted monomer could not be removed because the olive oil was too viscous for such small and complex structures.

After visual inspection of the samples, it thus was found that washing routines No. 1 and No. 3 rendered the best results. Both routines contain sonication in isopropanol. The only difference is the rinsing after the sonication, which is done with isopropanol and water for routine 1 and 3, respectively (see Table 5).

Based on these findings, the washing routine was further improved. The most advantageous washing routine was found to be washing the printed objects with isopropanol directly after printing and sonicate them as long and as many times as needed until the isopropanol remained transparent. After extensive ultrasonication, both rinsing with either isopropanol or isopropanol followed by water worked well. The latter yielded slightly better results because less salt was crystallized on the surface upon drying.

In a particularly advantageous washing routine, the objects are washed in isopropanol for 3 to 4 times to remove the samples from unreacted monomer and lose salt particles. After ultrasonication in isopropanol for 6 min and removal from the ultrasonic bath, the objects were washed again with isopropanol, followed by water, to remove remaining salt particles on the surface. A final rinsing with isopropanol prevents recrystallization of dissolved NaCl traces on the sample surface. The objects were then left upside down for drying at ambient temperature. It is also advantageous to dry complex structures in an air stream after the last rinsing.

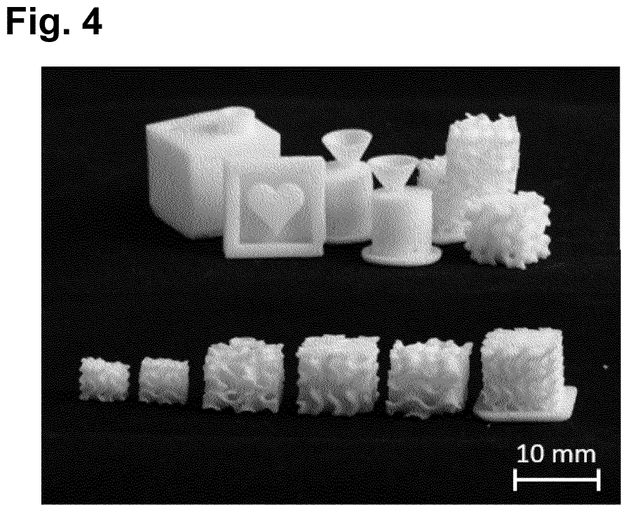

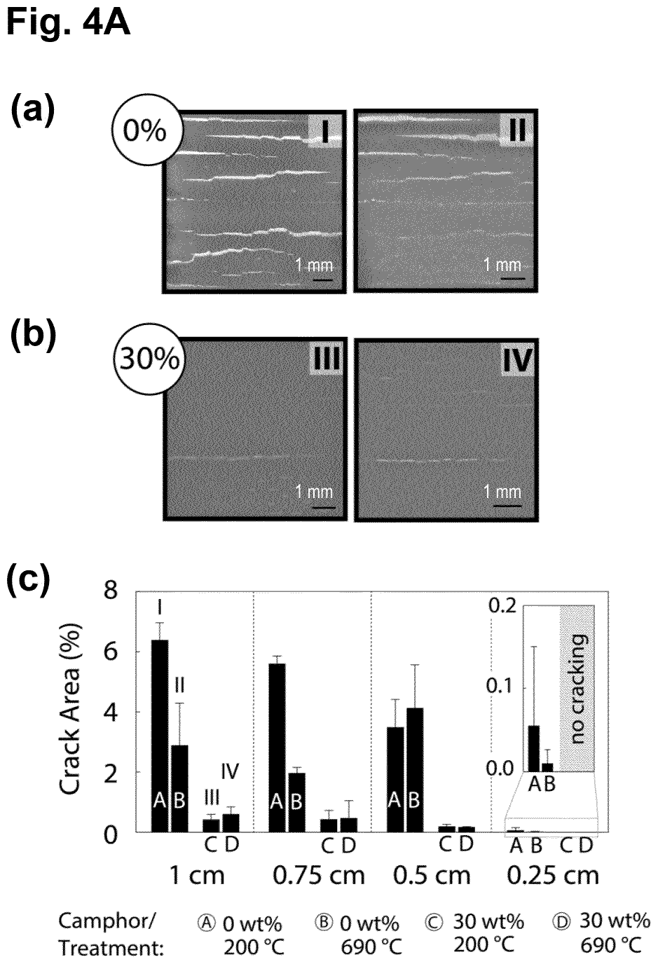

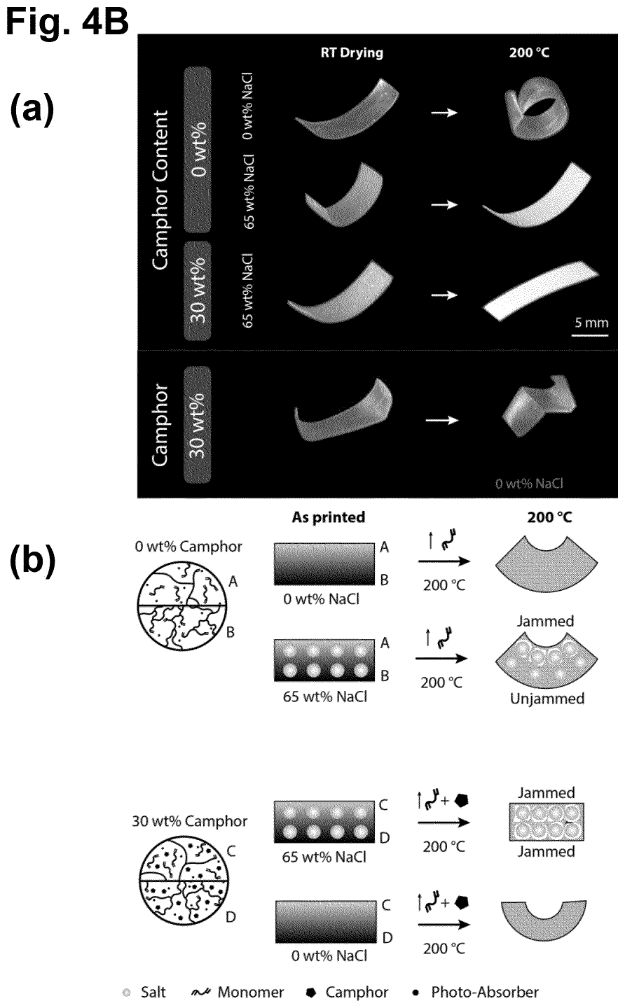

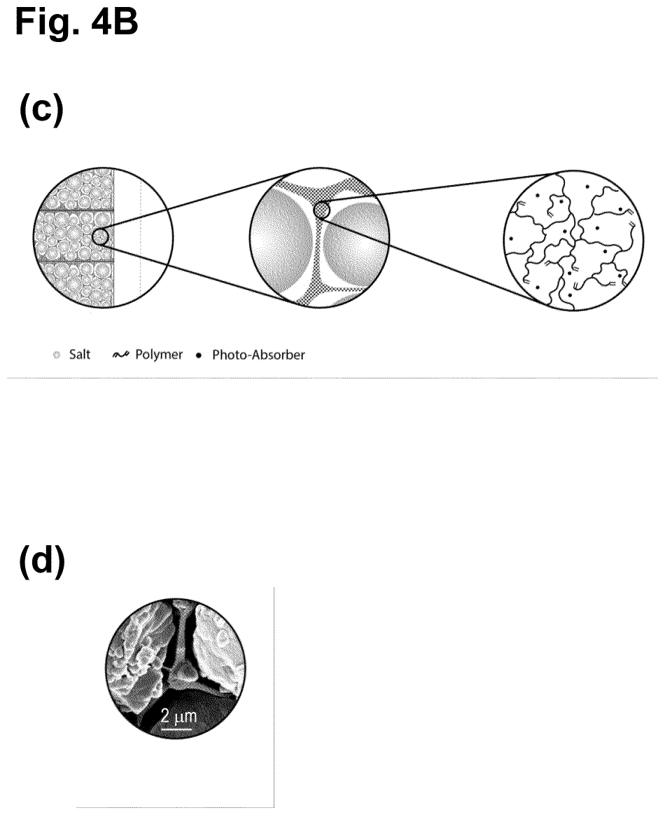

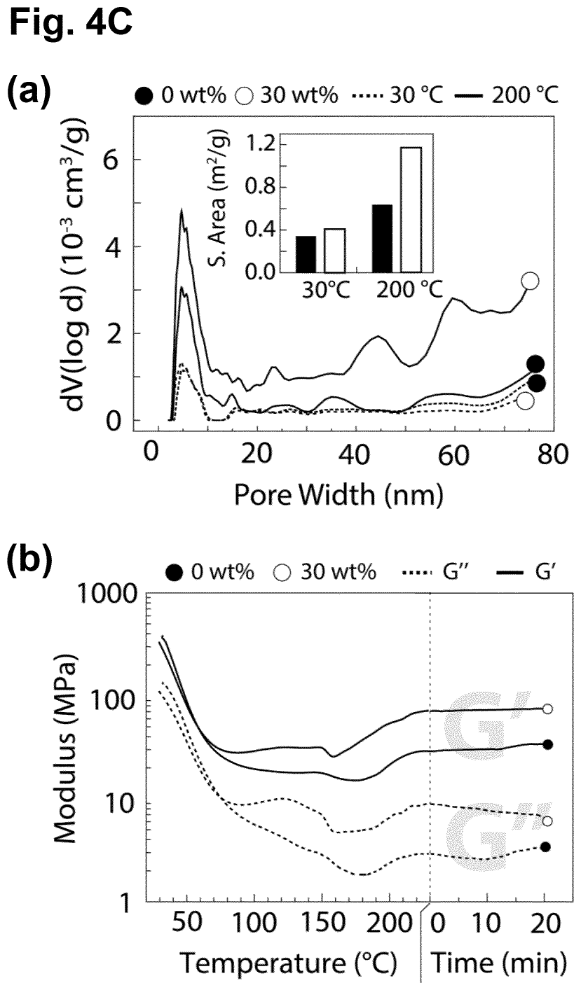

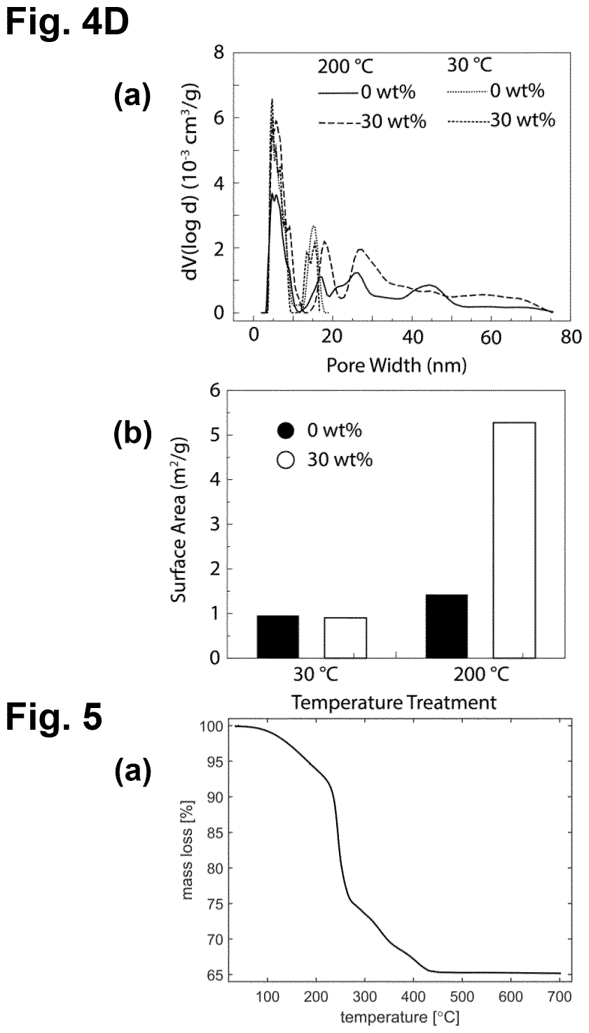

With such a washing method, defect-free objects as shown in FIG. 4 were obtained.

### Crack Formation

Crack formation can occur during and after various steps of the manufacturing process. Particularly during the debinding and sintering steps, the green bodies and binderless bodies are subject to thermal and mechanical stress, which can lead to crack formation. Cracks often are oriented along the printing planes, as was determined by 3D printing sample objects in different spatial orientations.

To reduce possible sources for crack formation, the above-discussed washing routine can be applied to remove unreacted monomer compounds prior to pyrolysis, in order to avoid thermally induced polymerization of remaining monomer. It has been experimentally shown that sonication of a sample object in isopropanol for 3×6 min strongly reduced crack formation, compared to sonication in isopropanol for 6 min.

Furthermore, post-curing with UV light can be used to react remaining monomer.

Finally, the fabrication of crack-free salt templates after calcination and sintering was experimentally found to require the presence of 30 wt % of camphor as diluent in the ink (with respect to monomer). To better understand the role of camphor in preventing cracking of the printed object during calcination, the microstructure of the polymerized ink after heat treatment at 200 and 690° C. was examined. In these experiments, cracks were quantified by image analysis of cracked area in printed cubes of different sizes imaged under transmitted light (FIG. 4A(a) 0 wt % camphor, FIG. 4A(b) 30 wt % camphor).

Cubes of different side length (0.25-1 cm) were printed as described previously. All cubes were printed with a targeted cure depth of 80 μm and post-processed as described above. No cracks were observed for any of the cubes after printing and cleaning. For the first heating step, the cubes were heated to 200° C. at 0.67° C./min in a Nabertherm LT furnace. The temperature was then held at 200° C. for 4 h, before cooling freely back down to room temperature (no active cooling or heating). For the second heating step, the samples were heated up to 200° C. at 3.3° C./min to allow for the continuation of the thermal treatment as described above. After each heating step, the samples were imaged optically in transmission on an optical microscope (Keyence, VHX-5000, Keyence). The images were analyzed by determining the pixel ratio of crack area (white in transmission micrographs) to cube side area in ImageJ (ImageJ2, version 2.3.0/1.53f). Since the samples are intrinsically anisotropic due to the layered build-up approach of 3D printing, only the planes of the cubes parallel to the z-axis were considered. These four sides showed the largest cracking due to the reduced material strength at the layer interface. All measurements were performed in triplicates.

In line with previous studies [24,60], the addition of 30 wt % diluent was found to significantly reduce cracking of the sample upon heating, decreasing the imaged cracked area of sintered 1 cm cubes from approximately 2.9% to values below 0.6% (FIG. 4A(c)).

Surprisingly, it has been discovered that the beneficial effect of camphor does not arise predominantly from its expected role in generating open pores upon sublimation to facilitate the removal of thermally degraded polymer at higher temperatures. Instead, experiments with the camphor-free inks show that extensive cracking is already observed when the sample is heated up to 200° C. (FIG. 4A), which is significantly lower than the thermal degradation temperature of the polymer (270-430° C.). By further increasing the temperature to 690° C., even a partial closure of cracks was detected, which reduce in area from 6.4 to 2.9% for 1 cm cubes without camphor.

Controlled experiments in single printed layers provide insightful hints into the possible effect of camphor in preventing extensive cracking during calcination at relatively low temperatures. The inks were individually exposed to light for 40 s in strips with a dimension of 20 mm×5 mm. No printhead was used, allowing the light to freely propagate through the ink or resin. The printed layers were wiped with a damp tissue with 2-propanol to remove unreacted ink. The samples were dried overnight (dried) and finally placed into the oven for the heat treatment at 200° C. The oven was heated from room temperature to 200° C. in 1 h, followed by an isothermal hold at 200° C. for another hour. Finally, the samples were left to cool back to room temperature. It is hypothesized that internal stresses manifested as warpage in these model experiments may cause cracking of the multi-layered printed object during thermal treatment.

Photographs of a polymerized sample without salt particles and camphor reveal strong warpage of the single layer upon drying at RT, an effect that becomes even more pronounced at 200° C. (FIG. 4B(a)). The same effect was found for samples with camphor but without NaCl (FIG. 4B(a)). This suggests the presence of a gradient in monomer conversion and cross-linking density across the thickness of the layer (FIG. 4B(b) (right)). Because of the lower illumination at the top of the layer (i.e. further away from the illumination source in the printing setup, this region likely contains a higher concentration of unreacted monomers compared to the directly exposed bottom side of the layer that is exposed to maximum dosage. Such a gradient in unreacted monomer concentration would translate into differential shrinkage of the layer during free monomer removal upon washing and drying, leading to the build-up of internal stresses and ultimately causes warpage.

The addition of salt particles to the camphor-free polymerized samples leads to even stronger warpage after room-temperature drying. Interestingly, the high initial warpage of such layer reduces significantly when the sample is further heated to 200° C. This effect is interpreted as a result of jamming of the salt particles at the not directly illuminated top side of the layer, which restricts the shrinking during monomer evaporation due to the formation of a compressed load-bearing particle network. This leads to preferential shrinkage of the directly illuminated bottom side of the layer, partially compensating for the strong initial warpage. Importantly, our experiments show that the initial warpage of the layer can be fully reversed when camphor is added to the formulation. It is assumed that sublimation of camphor leads to sufficient shrinkage of the sample, such that particles at both sides of the layer become jammed. By providing a locking mechanism that is triggered via the sublimation of camphor, the salt particles prevent the build-up of differential stresses across the sample cross section that cause cracking in the thermally treated printed objects.

SEM images from samples dried for 20 h at 200° C. confirm the strong shrinkage of the polymer phase and the formation of pores between the salt particles after the sublimation of the diluent (FIG. 4B(d)). Nanoporosity, pore-size distribution and surface area were determined by nitrogen gas sorption at 77 K on a Quantachrome Autosorb iQ. The samples were outgassed for at least 24 h. The outgassing temperature was set to 30° C. for samples without any thermal treatment, or to 80° C. for previously heat-treated samples. Density functional theory (DFT) analysis was used to determine the pore size and volume. The calculations were performed using a non-local DFT (NLDFT) sorption model based on N₂ adsorption on cylindrical silica pores at 77 K. The surface area was determined by the Brunauer-Emmett-Teller (BET) method. With the help of nitrogen gas sorption analysis, it was found that this porosity on the micrometer scale is complemented by presence of nanoscale pores in the range of 2-10 nm, as well as larger pores ranging from 20 to 75 nm in size (FIG. 4C(a)). Samples prepared with camphor show higher porosity and surface area compared to reference samples without camphor and those with removed camphor upon heat treatment at 200° C. (FIG. 4C(a) and FIG. 4D). Because smaller nanopores are already present in camphor-free samples kept at 30° C., the smaller nanopores are presumably part of the polymerized resin. Instead, the larger nanopores only appear upon drying at 200° C. and are therefore clearly associated with the removal of the diluent from the camphor-containing sample. Finally, the pores at the micrometer scale are generated due to the contraction of the polymer phase upon diluent sublimation. These pores are likely to facilitate the thermal decomposition of the polymer at higher temperatures.

The proposed microstructural locking effect induced by the removal of camphor is reflected in the evolution of the mechanical properties of the printed object upon heating (FIG. 4C(b)). In order to perform dynamic mechanical analysis (DMA), rectangular samples with a length of 40-45 mm and a width and height of 25 mm were printed. The samples were printed on supports to eliminate any influence of increased light exposure of the first layers during printing. The measurements were conducted on a TA instrument rheometer (ARES-G2, TA Instruments) in nitrogen atmosphere by applying a constant torsional frequency of 3 Hz and an oscillation strain of 0.1%. The rheometer was equipped with an environmental test chamber and a torsion fixture for rectangular samples. In a first interval, the temperature was ramped at 2° C./min from 30° C. to 230° C. The temperature was further kept constant at 230° C. for 20 min in a second interval. Over the course of both intervals, the storage (G′) and loss (G″) moduli were monitored. After an initial softening during heating to 100° C., the storage modulus (G′) of the camphor-containing composite was found to increase from 31 to 75 MPa when the temperature was raised from 150 to 230° C. This 1.5-fold increase in modulus contrasts with the 0.9-fold enhancement observed for the camphor-free sample within the same temperature interval. The higher stiffness of the composite prepared with camphor probably results from the formation of the load-bearing network of salt particles upon removal of the diluent phase.

### Debinding and Sintering

After 3D printing and subsequent washing the object, the polymer binder matrix holding the salt particle structure together has to be pyrolyzed to obtain the binderless body. The binderless body has to be sintered, in order to connect the particles to obtain sufficient mechanical stability for the future use as a mold.

Pyrolysis and sintering were carried out in one cycle using a conventional electrical furnace (HT 08/17, Nabertherm GmbH). However, the debinding and sintering can also be conducted in separate steps. Debinding is carried out in ambient atmosphere. Sintering can be conducted in inert, reducing or ambient atmosphere. Depending on the slurry composition and the sintering temperature, atmosphere and time, the above mentioned heat treatment will result in a partially to fully dense sintered body.

The debinding step is critical because all of the organic material has to be pyrolyzed and leaves the ceramic material as gas phase, which subjects the green body to mechanical stress and may lead to crack formation.

A heating rate that is too high does not leave enough time for the volatile decomposition products to diffuse out of the material, resulting in pressure buildup, which leads to mechanical defects such as cracking or delamination.

It is also advantageous to obtain a mold that is sufficiently dense. For a proper infiltration of casting materials with low viscosity into the molds, the mold or at least its surface has to be dense or not wettable by the casting material. Otherwise, the liquid casting material will penetrate into the NaCl microstructure resulting in poor surface quality and impeding the dissolution of the NaCl in the subsequent manufacturing step.

The optimal conditions for debinding cycle are typically determined by thermogravimetric analysis (TGA, STA 449 C, Netzsch-Gerätebau GmbH).

Isothermal treatments are usually performed at temperatures at which the differential thermal analysis (DTA) shows peaks, i.e. when the decomposition processes occur. For objects with large cross sections, diffusion of decomposition products out of the structure is hindered, resulting in stresses. A way to reduce these stresses is to add a suitable non-reacting diluent. This reduces the overall concentration of photosensitive material and leaves some voids or channels prior to the decomposition of the polymeric material, since the diluent evaporates or decomposes at lower temperatures. Moreover, remaining unreacted monomer can induce mechanical stress during debinding due to shrinkage upon heat-induced polymerization of the remaining monomer.

To find suitable conditions for the debinding and sintering cycle adjusted to the given system, several characterization methods were applied. The influence of the AOT concentration, sintering time, and sintering temperature on the final density has been investigated.

The sample objects were cylinders with a diameter of 3 mm and a height of 5 mm and were prepared from composition Variant b, Table 4.

Shrinkage during sintering was assessed by measuring the weight, height and diameter of the green body and comparing this data to the same sample after sintering. Several parameters were investigated, namely the sintering time (i.e. duration of isotherm at sintering temperature; 0 h, 4 h, 8 h), the sintering temperature (650° C., 690° C., 730° C.) and the surfactant concentration (0.1 wt % to 5 wt % AOT during salt particle preparation).

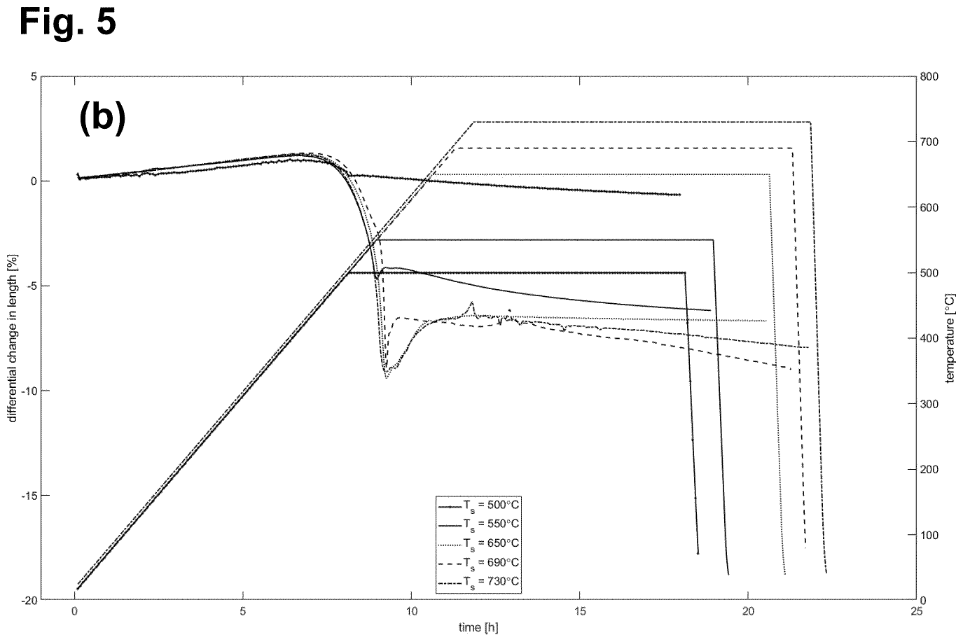

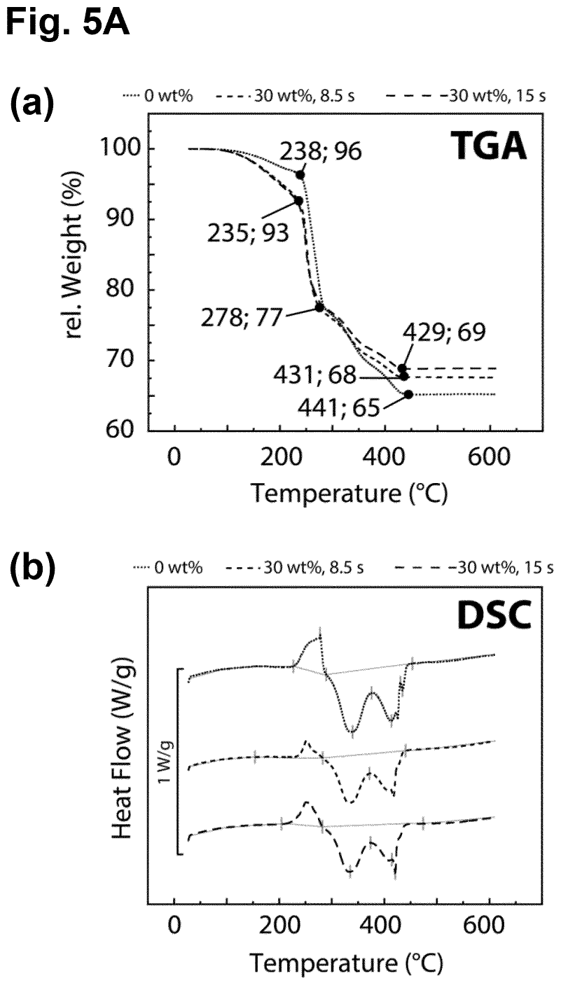

Thermogravimetric analysis was carried out for assessing suitable conditions for debinding, namely determining temperatures of highest mass loss, in order to determine the temperature of isotherms for the debinding profile. The decomposition reaction and products were not studied in detail. As depicted in FIG. 5(a), there is a large mass loss of the sample objects around 200° C. for samples containing 30 wt % camphor, 0.125 wt % UV blocker and 3 wt % Sudan I. The mass further drops to 65% between 250° C. and 450° C. At higher temperatures, no further mass loss is registered, indicating the completion of the decomposition reaction.

A similar analysis, combined with dilatometry measurements, was performed on a TGA/DSC 3+, Mettler Toledo with printed cylinders with 30 wt % camphor or without (0 wt %) camphor, 0.05 wt % Sudan I and 2 wt % BAPO (FIG. 5A(a)). Printed samples were weighted in a lid-covered Al₂O₃ crucible (70 μL) while heated between 25° C. and 600° C. at a heating rate of 2° C./min in air. Pyrolysis and sintering were further performed in a single temperature treatment in a Nabertherm LT furnace. In a first step, the samples were heated from room temperature to 200° C. at 0.67° C./min. The temperature was then kept at 200° C. for 4 h to remove volatile organic residues such as the non-reacted monomer and the diluent. The temperature was afterwards further increased to 350° C. at 0.25° C./min and held at 300° C. for 4 h. To complete the pyrolysis of the polymerized polymer matrix, the samples were further heated to 450° C. at 0.25° C./min and kept constant thereafter for another 4 h. Finally, sintering was performed by increasing the temperature to 690° C. at 1° C./min. Full densification was obtained by holding the sintering temperature for 4 h. The measurement was performed with an empty reference crucible, and calibrated with a blank curve measurement. The TGA data show that samples with diluent undergo a mild weight loss of 7.4% when heated up to about 230° C., which is followed by a sequence of mass drops reaching in total 32.3% at 430° C. Combined with differential scanning calorimetry (FIG. 5A(b)), these results suggest that small amounts of unreacted monomers and camphor are removed from the sample below 230° C., followed by the evaporation of unreacted monomers up to 280° C. Further heating above this temperature results in the thermal decomposition of the polymerized organic phase. Comparison between samples with and without 30 wt % camphor indicates that this compound sublimates from the polymerized resin at temperatures between 85° C. and 240° C.

In order to find suitable sintering parameters for this system, dilatometric data was acquired, providing information about the length change of the sample over time for a distinct temperature profile. FIG. 5(b) shows the differential change in length for sintering temperatures 500° C., 550° C., 650° C., 690° C., 730° C. Pyrolysis and sintering were carried out in one cycle using a conventional resistance furnace (HT 08/17, Nabertherm GmbH).

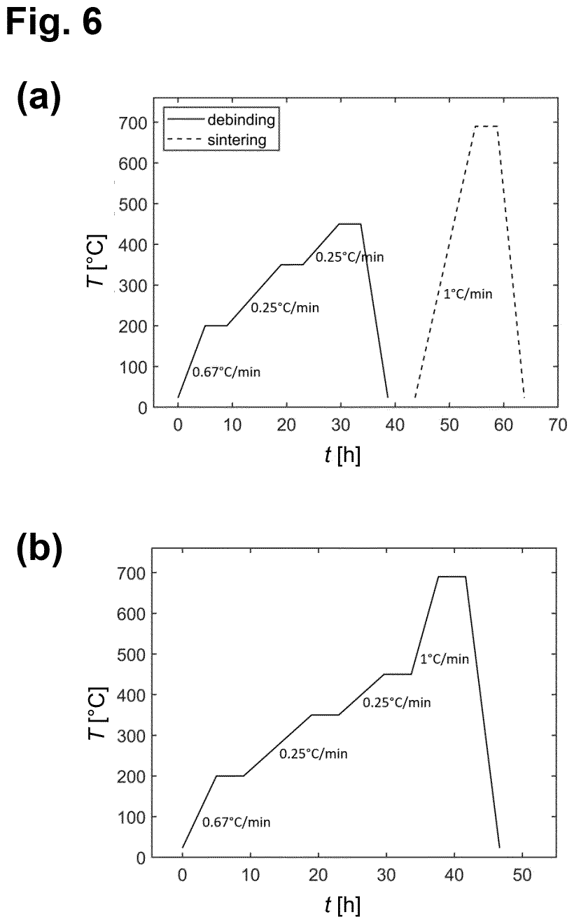

The temperatures at the mass loss peaks were chosen as isotherms in the pyrolysis cycle in order to assure complete polymer removal. The corresponding debinding cycle is shown in FIG. 6(a). The isotherms chosen for the debinding cycle are 200° C., 350° C., and 450° C., with the increase rates listed in FIG. 6, and, with a holding time of 4 h at each isotherm. This gives the pyrolysis reaction and the resulting decomposition products sufficient time for diffusing out of the sample, without risking pressure buildup and crack formation. Above 450° C., the decomposition is completed because no further mass is lost, as shown in FIG. 5(a).

The sintering can be carried out separately, as indicated by the dashed line in FIG. 6(a), with a sintering isotherm of 690° C. and a holding time of 4 h. More advantageously, the debinding and the sintering steps are combined to one single process, as shown in FIG. 6(b).

All the differential changes in length follow the same curve up to approximately 9 h, as displayed in FIG. 5(b). A relative decrease in length reflects an increase of the density. At a sintering temperature of 500° C., the NaCl template hardly densified. Increasing the sintering temperature to 550° C. resulted in a larger change in length at around 9 h. However, the largest change in length was realized with temperatures of 650° C. and higher.

The influence of the sintering temperature and sintering time for different AOT surfactant concentrations on the relative density (in relation to the maximal density of crystalline NaCl) and shrinkage was studied.

The density of the sintered samples was determined with the Archimedes principle (Used device: XS205 DualRange, Mettler-Toledo GmbH). Ethylene glycol was used as the medium to measure the buoyancy of the samples, and hence determine their density. Using the density of NaCl (2.16 g/cm³), the relative density of the sintered sample was calculated.

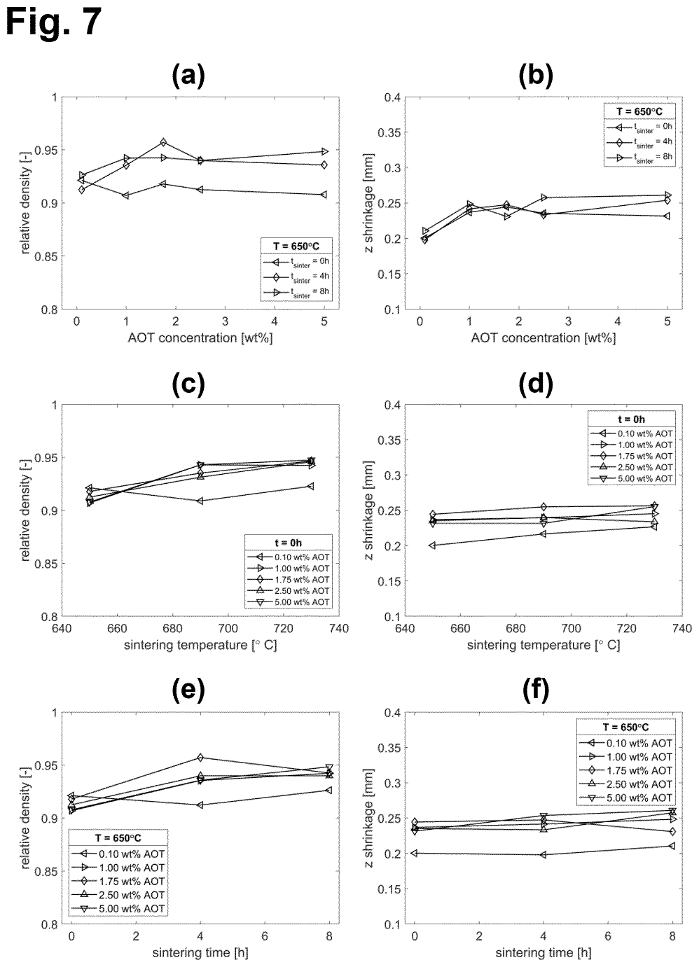

The experimental data in FIGS. 7(a) and 7(b) show that for low sintering temperatures and low AOT concentrations the relative density as well as the shrinkage in z direction is largely determined by the applied sintering time. Similarly, keeping the sintering time at 0 h (i.e. no isotherm at the sintering temperature), an increase of the sintering temperature results in an increase of the density for all AOT concentrations, as shown in FIG. 7(c). It should be noted that a sintering time of 0 h does not mean that no sintering takes place, but that the holding time at the sintering isotherm is 0 h. Sintering will nevertheless take place to a certain degree close to the sintering temperature, namely during the increase of the temperature toward the sintering isotherm and the subsequent decrease in temperature.

z-shrinkage only exhibits a trend for the lowest AOT concentration and remains nearly constant for higher AOT concentrations, as can be seen in FIG. 7(d). The surfactant concentration seems to have the largest influence, as shown in FIGS. 7(c) to 7(f).

It is assumed that Na₂SO₄ acts as sintering aid, yielding higher final densities compared to the influence of sintering temperature Tₛ and sintering time tₛ. One decomposition product of AOT during pyrolysis is sodium sulfate Na₂SO₄, which together with NaCl forms a eutectic composition.

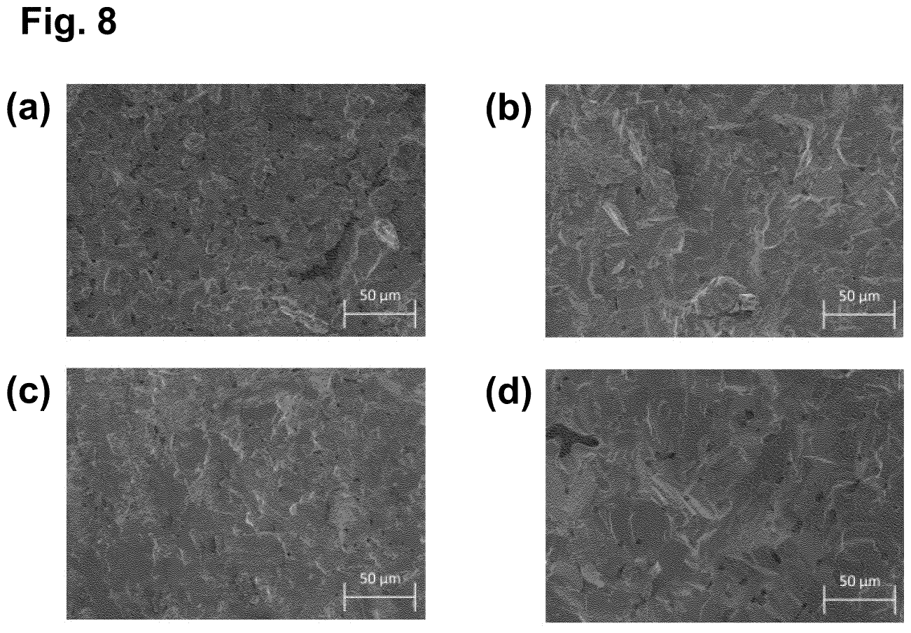

FIG. 8 supports the same findings. Scanning electron microscopy (SEM) was used to obtain morphological information of the resulting microstructure surface of the NaCl molds. Imaging was performed at 5 kV with an InLens detector. The samples were mounted on carbon stickers and sputtered with 6-10 nm Pt to enable good sample conductivity.

FIGS. 8(a) (with 0.1 wt % AOT) and 7(c) (with 5 wt % AOT) show the surface prior to sintering (tₛ=0). The increased concentration of AOT surfactant in the green body leads to a denser, less porous surface already after the debinding step. Similar effects take place when sintering is applied, as shown in FIGS. 7(a) (with tₛ=0 h) and FIG. 7(b) (with tₛ=8 h) or when the sintering temperature is increased, as shown in FIGS. 7(a) (with Tₛ=650° C.) and FIG. 7(d) (with Tₛ=730° C.).

The defect-free infiltration of NaCl printed templates requires molds with liquid-tight surfaces. However, the pressureless sintering of pure NaCl to high density is very challenging [61]. Dense structures can be obtained by optimizing any of these three parameters. However, the increase in AOT concentration renders the densest microstructure (FIG. 8(c). This supports the result from the dilatometry measurements. For a higher concentration of AOT (1.75 wt % AOT was used for dilatometry), the sintering time and temperature do not considerably influence the density. This result shows that dense NaCl molds can be manufactured at lower sintering temperatures and shorter sintering times, which is advantageous in view of energy consumption and manufacturing time. For an AOT surfactant concentration of 1.75 wt %, a dense microstructure can be achieved at 650° C. for a sintering time of 4 h.

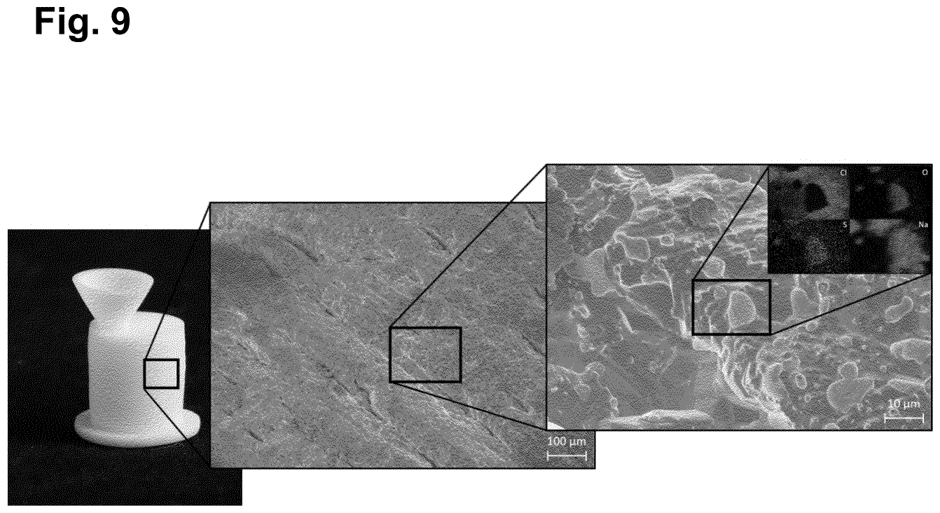

Since it is assumed that sodium sulfate Na₂SO₄ is responsible for the enhanced sintering, energy dispersive X-ray spectroscopy (EDS) measurements were conducted on the particles found on the NaCl mold surface to prove its presence (5 kV, LEO 1530, Zeiss). In FIG. 9, it can be clearly seen that the particle of interest contains Na, O, S, but no Cl. This supports the assumption that Na₂SO₄ is present in the system and that it acts as sintering aid.

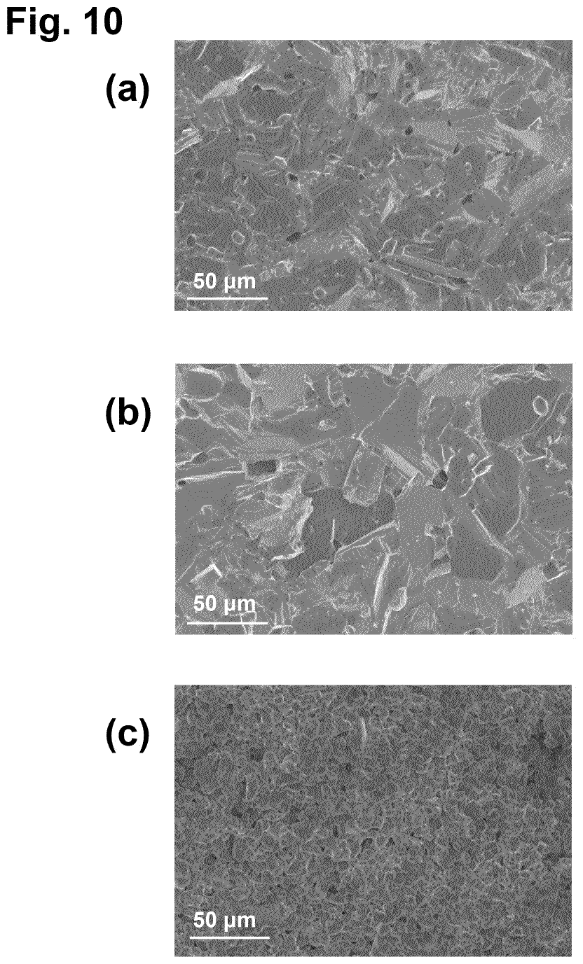

Further evidence of the sintering aid like behavior of AOT due to its decomposition into Na₂SO₄ is provided in FIG. 10. Slurries without UV blocker were cast and cured under UV light. The samples were heat treated and sintered at 690° C. for 4 h. FIG. 10(a) depicts the microstructure of a slurry prepared with 1.75 wt % AOT (with respect to NaCl). A very similar, dense microstructure is obtained for slurries with 0 wt % AOT, but with 1.9 wt % Na₂SO₄ (with respect to NaCl). In contrast, samples which contain neither AOT nor Na₂SO₄ show high porosity (FIG. 10(c)).

The weight of the sintered samples was recorded in dry (w_{d}), saturated (wₛₐₜ) and fully immersed (w_{A}) states. Soaking and immersion were performed in NaCl-saturated ethylene glycol. The open porosity (f) was then calculated using the relation [cf. 62]:

$$f = \frac{{w}_{sat} - {w}_{d}}{{w}_{sat} - {w}_{A}} .$$

Combined with SEM imaging, our measurements revealed that samples processed without additives had an interconnected pore network with a high remaining porosity of 11.4 vol % and small grain size (FIG. 10(c). Such a pore network would be penetrated during material infiltration and hence distort any desired shape. It was found that the porosity could be strongly reduced to 2.1 vol % and 1.8 vol % by the addition of AOT (FIG. 10(a)) or Na₂SO₄ (FIG. 10(b)), respectively, to the initial ink.

These findings further support the hypothesis, that AOT has a dual role as particle dispersant during printing, and as sintering aid after its decomposition into Na₂SO₄.

### Casting

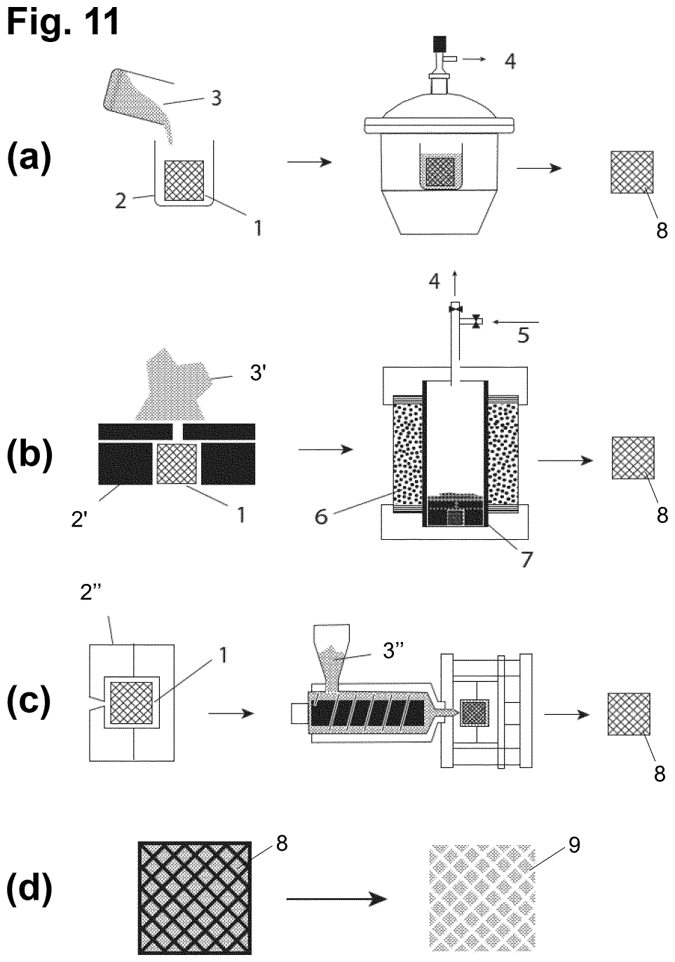

The infiltration or coating of the samples depend on the infiltration material and geometries. In general, the material is infiltrated by a pressure difference, with or without simultaneous temperature aid to melt a material and/or lower its viscosity. An illustrative description of varying infiltration processes is shown in FIG. 11.

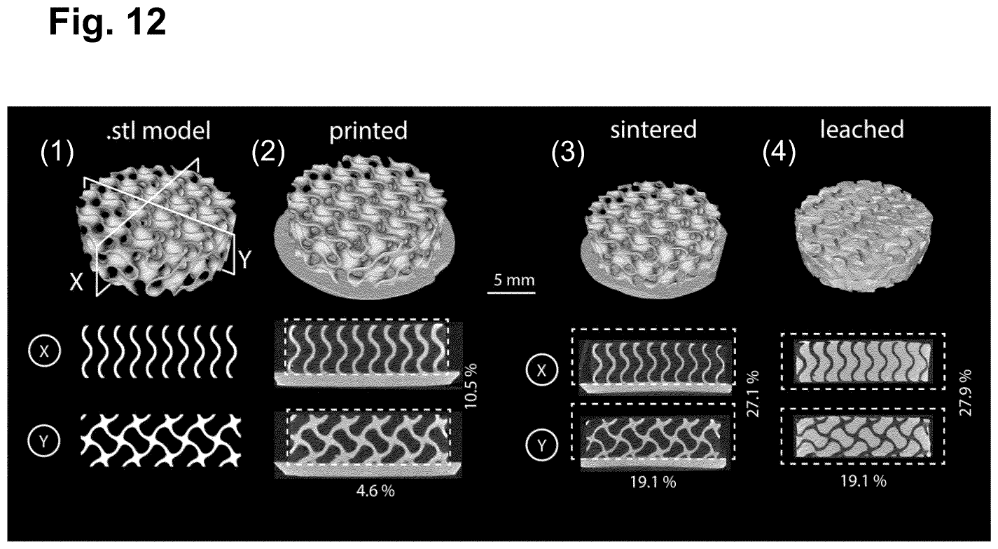

The ability to obtain crack-free salt objects after the drying and sintering steps opens the way towards the manufacturing of complex-shaped structures for a variety of materials by simple infiltration and leaching of the salt template. To illustrate this process, a salt object with a complex gyroid geometry was generated and used it as a template to create polymer scaffolds with unique three-dimensional architecture. The fidelity of the manufacturing process is evaluated by performing microcomputed tomography (microCT) of the salt-based object after each of the printing, sintering and infiltration steps (FIG. 12). MicroCT was performed on one sample of a dried and sintered gyroid template, as well as on the corresponding casted and leached silicone scaffold, using a MicroCT100 instrument (Scano Medical AG). The scans were performed with an energy level of 55 kVp (peak kilovoltage), an intensity of 8 W, an exposure time of 139 ms and a nominal resolution of 10 μm. The data was analyzed in 3D Slicer (version 4.11.20210226, http://www.slicer.org).

Digitally visualized cross-sections obtained from the microCT of the object demonstrate that the manufacturing process preserves with high fidelity the morphology of the original digital design throughout each of the multiple steps. Importantly, the shrinkage of the object along the process needs to be considered in the original design to reach the desired final dimensions. Because of the layer-by-layer nature of the printing process, the object was found to shrink predominantly along the vertical (z) direction during the post-printing drying and sintering steps. This is well in-line with previous sintering studies of particle-based stereolithography printing [63,64]. Linear shrinkage in the z-direction reached 27.9% after complete heat treatment, as opposed to a value of 19.1% along the x- and y-directions (FIG. 12, bottom). The observed shrinkage can be beneficial for the fabrication of complex structures with dimensions below the resolution limit of the printer.

The open porosity of the chosen gyroid structure allows for facile infiltration of the sintered salt template by simple casting of a thermal curable resin or injection-molding of a molten polymer.

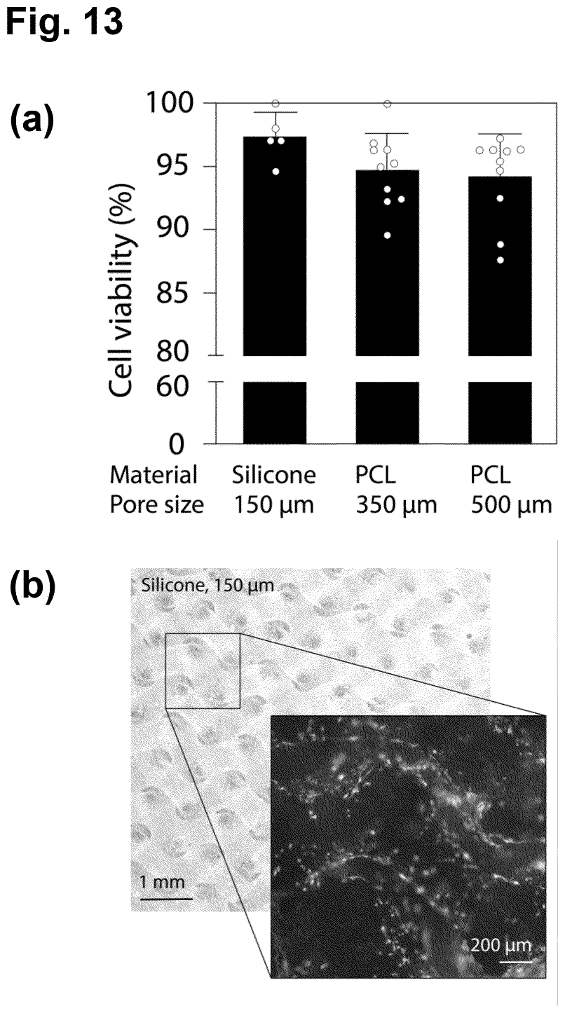

The compatibility of our developed salt templates with these two infiltration approaches is demonstrated (FIG. 13(a)), using commercially available biomedical silicone resin and thermoplastic polycaprolactone (PCL). MicroCT images of the resulting infiltrated and leached silicone scaffold indicate minimal shrinkage of the object and the formation of a pore-free polymer phase within the interstices of the leached salt structure. Upon leaching in water at room temperature, porous silicone and PCL scaffolds with exquisite and complex three-dimensional architecture (cf. bright field image in FIG. 13(b)) were obtained.

Low viscosity materials such as epoxy, radiation curable monomers or low viscosity silicones were infiltrated by assistance of vacuum (FIG. 11(a)). In this case, the salt mold 1 is placed in a glass beaker 2, which is further filled with a low viscosity material 3. This set-up is further transferred into a desiccator for vacuum 4 application. The vacuum is maintained until no gas bubbles appear from the infiltrated material surface. The set-up is further removed from the desiccator for curing at elevated temperature or irradiation curing.

For metal infiltration, the salt mold 1 is placed into an outer mold 2′ e.g. made from graphite, which in turn is placed in a graphite tube 7 inside a heating cylinder 6. This gas-tight set-up is placed inside an oven. After vacuum 4 application, the set-up is heated, and the molten metal 3′ is infiltrated by the application of inert gas 5 pressure. Molten magnesium metal was casted at 700° C. and an argon pressure of 5 bar, after vacuum application (FIG. 11(b)).

For more viscous materials, injection molding can be used for infiltration. Injection molding can be performed by a small hand-operated injection molding device, or by an industrial injection molding machine. For both cases, the salt mold 1 is placed into a metallic outer mold 2″. Polycaprolactone 3″ (PCL, M_{w}=40′000) was injection molded at a pressure of 2 bar. Low molecular weight polypropylene (PP) was injection molded at 60 bar (FIG. 11(c)).

Scaffolds with such digitally programmable pore size and morphology are highly desired for the ingrowth of cells and tissues in biomedical applications. Since the material of interest is directly infiltrated into the salt, it does not contain unreacted monomers or additional chemicals typically required for the light-based printing of polymers. This beneficial feature allows for the fabrication of complex-shaped biocompatible structures without toxic residues that could be harmful for living cells. While salt templating is often used to fabricate bio-scaffolds, the poor geometrical control over the pore structure of existing processes challenges the cell seeding efficiency and vascularization thereof.

To demonstrate the suitability of the proposed process in fabricating scaffolds for biomedical applications, the viability of pre-osteoblast cells on silicone and PCL scaffolds displaying a gyroid structure with distinct pore sizes in the range 150-500 μm was measured (FIG. 13). Mouse pre-osteoblast cells (MC3T3-E1, passage number 31) were obtained from University of Zurich, Zurich, Switzerland. First, 5×10⁴ cells were seeded onto the scaffolds that were beforehand immersed in a fibronectin solution (Human Plasma Fibronectin Purified Protein, Sigma-Aldrich, FC010, 0.01 mg/ml in sterile PBS) for 3 h. After allowing cells to attach for 2 h, growth medium composed of MEM α without ascorbic acid (Gibco, A1049001), 10% fetal bovine serum (Gibco, 26140079) and 1% antibiotic-antimycotic (Gibco, 15240062) was added to the cell culture. After 2 days of culture, the scaffolds were washed with PBS and the cells were stained for 10 min in 0.5 μL/mL calcein-AM and 2 μL/mL ethidium homodimer-1 from the Live/Dead assay kit (Invitrogen, L3224). This was followed by cell fixation in 4% paraformaldehyde (Santa Cruz Biotech, 281692) for 15 min before washing again with PBS. Finally, confocal laser scanning microscopy (Zeiss, LSM 780 upright) was used to visualize the cells. Cell viability was calculated as the number of live cells divided by the total number of cells. Before cell seeding, the scaffold surfaces were pretreated to promote cell adhesion with either silk fibronectin or sodium hydroxide for the silicone and PCL scaffolds, respectively. The results obtained after 2 days of culture show cell viability higher than 94% for all the scaffolds tested, confirming the cytocompatibility of the polymer structures and their suitability for in vitro cell culture. In light of the latter, pre-osteoblasts cells were observed to spread homogeneously over the scaffold surface and to easily penetrate into the open porous architecture.

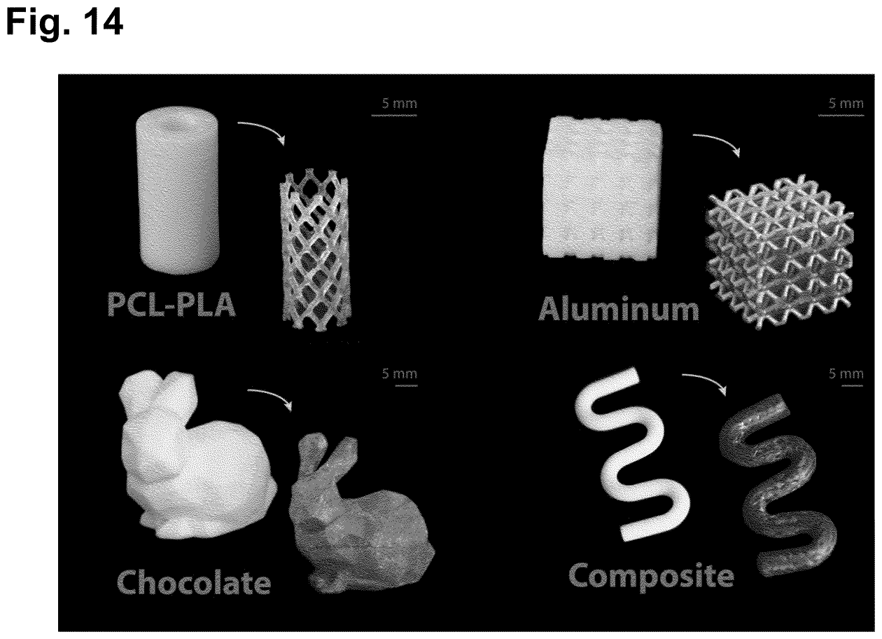

Beyond silicone and PCL, a broad range of materials can be shaped into complex architectures using the proposed manufacturing platform. This universal nature is demonstrated by creating intricate 3D structures from various materials as diverse as chocolate, aluminum, magnesium, carbon fiber composites and degradable polymers (FIG. 14 and FIG. 15). The process diversity is further demonstrated by shaping these materials through infiltration into salt molds, intricate salt templates of by warping of salt cores.

Complex-shaped structures were generated by casting or infiltration of the salt templates following different procedures depending on the chemical and physical nature of the infiltrating material. Polymeric stent—The pre-polymers of randomly polymerized D,L-lactide (DLLA) and ε-caprolactone(CL) was prepared as previously reported [37]. In short, a four-armed copolymer of 15000 g/mol and a linear copolymer of 650 g/mol were synthesized using a monomer ratio CL/DLLA of 7/3 and 2/2, respectively. The obtained copolymers were mixed by combining 75 wt % four-armed and 25 wt % linear molecules. This pre-polymer mixture and 3 wt % of initiator (TPO-L, Speedcure) were heated to 60° C. to decrease the viscosity and facilitate homogenization. The resulting photosensitive resin was manually pressed into the sintered salt mold and cured by UV-light (Omnicure S1000, Lumen Dynamics) for 3×10 min. The mold was finally dissolved in water. Aluminum lattice—The salt templates were infiltrated as described previously [65]. A refractory crucible with the NaCl templates and a piece of AlSi 12.6 wt % aluminum alloy was heated to 710° C. under vacuum. After a holding time of 3 min, an Ar pressure of 15 bar was applied to enable metal infiltration. Finally, the directionally cooled samples were cut to gain access to the NaCl templates, which were subsequently leached in water. Chocolate bunny—Swiss dark chocolate was molten, filled into the salt mold and placed into the fridge for full solidification. The mold was dissolved in ice-water to prevent the chocolate from re-melting. Carbon fiber composite—The printed and heat-treated NaCl cores were wrapped with two layers of a braided carbon fiber sleeve (5 mm diameter, Suter Kunststoffe AG), infiltrated with a low viscosity epoxy resin (EPIKOTE™ Resin MGS®, Suter Kunststoffe AG) and cured at room temperature with a shrink tape for compression (R&G Filament 160, Suter Kunststoffe AG, Switzerland) before final leaching in water.

The complex mold geometries enabled by light-based printing allows to shape these materials into 3D structures that would be very challenging to achieve using conventional manufacturing technologies. For example, biodegradable high molecular weight poly(D,L-lactide-co-8-caprolactone) methacrylate (poly(DLLA-co-CL) MA) copolymers that are too viscous for light-based direct printing [37] were easily molded into stent-like geometries using printed salt templates (FIG. 4c, top left). In another instance, lightweight aluminum lattices and bioresorbable magnesium with intricate porous architectures were manufactured by molten metal infiltration of salt templates at 710° C. and 700° C., respectively. Additional examples are displayed here (FIG. 15), including hierarchically porous poly(ε-caprolactone) (PCL, FIG. 15(a)), magnesium (FIG. 15(b)) and polystyrene (FIG. 15,c). These materials are all challenging to print directly at high resolution but have a high potential for biomedical applications such as 3D cell cultures (PCL and PS) and biodegradable bone scaffolds (PCL and Mg). The selected examples demonstrate the high versatility of the process, which enables structuring materials from room temperature to >700° C., including metals with high reactivity such as magnesium. Moreover, the templating process may be combined with traditional pore-forming techniques, such as conventional salt leaching, to create PCL scaffolds with unique hierarchical porosity (FIG. 15(a)). The hierarchical porosity was achieved by combining conventional salt leaching for the micron-sized porosity, with our NaCl templates for the macroscopic architectured lattice structure. 3 g of PCL were dissolved in a solution of 4 mL of chloroform and 1 mL ethanol. 13.2 g ball-milled and functionalized NaCl particles (1.75 wt % AOT) were thoroughly mixed into the PCL solution. The particles had previously been prepared as described in the Materials and Methods section. The dispersion was manually pushed into the salt templates and left to dry for 2 days, followed by leaching of the NaCl particles and templates in tap water.

This plethora of examples demonstrate the potential of this light-based printing technology in leveraging salt templating for the shaping of so far inaccessible materials and geometries.

All methods result in an infiltrated body 8, which is further leached in aqueous solution to obtain the final part 9.
1: NaCl mold/template; first mold; 2, 2′, 2″: second, outer mold; 3, 3′, 3″: material for infiltration or casting or molding; 4: connection for vacuum application; 5: inert gas inlet for pressure casting; 6: insulation material (e.g. alumina wool); 7: crucible (e.g. graphite); 8: infiltrated template mold; 9: final part.

For carbon fiber composites, the sintered articles are wrapped with the carbon fabric and coated with an epoxy resin. The fabric was further impregnated with the epoxy by vacuum application in a vacuum bag.

Upon solidification of the materials or epoxy, the sintered salt article was removed by immersion in a polar solvent, such as water. The demolding is further facilitated by ultrasonication, solvent jetting or mechanical force.

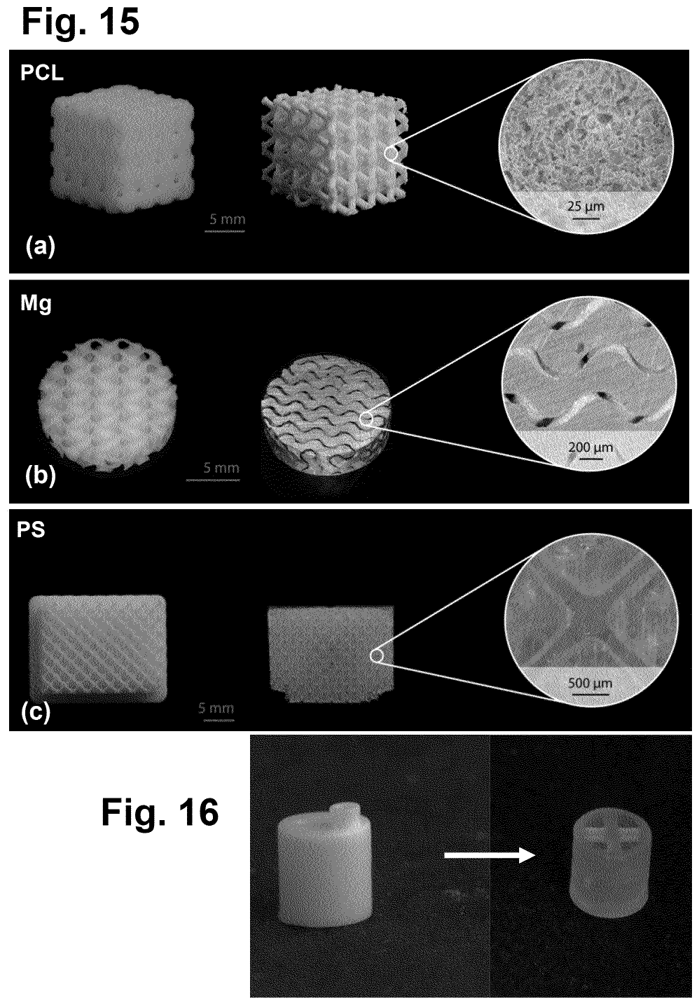

An example of a cast epoxy resin object is shown on the right side of FIG. 16, while on the left side, a NaCl mold according to the invention shown, with which the epoxy resin object has been cast.

The present invention is not to be limited in scope by the specific embodiments de-scribed herein. Indeed, various modifications of the present invention, in addition to those described herein, will be apparent to those skilled in the art from the foregoing description and accompanying drawings. Thus, such modifications are intended to fall within the scope of the appended claims. Additionally, various references are cited throughout the specification, the disclosures of which are each incorporated herein by reference in their entirety.

### REFERENCES

[1] V. Karageorgiou, D. Kaplan, Biomaterials 2005, 26(27), 5474-5491
[2] Y. Conde et al., Adv. Eng. Mater. 2006, 8(9), 795.
[3] R. Nazarov, H.-J. Jin, D. L. Kaplan, Biomacromolecules 2004, 5, 718.
[4] Q. Hou, D. W. Grijpma, J. Feijen, Biomaterials 2003, 24, 1937.
[5] A. H. Brothers, D. C. Dunand, Adv. Mater. 2005, 17, 484.
[6] R. Goodall, A. Mortensen, Adv. Eng. Mater. 2007, 9, 951.
[7] K. Lietaert et al., J. Magnesium Alloys 2013, 1, 303.
[8] T. A. Schaedler, W. B. Carter, Annu. Rev. Mater. Res. 2016, 46, 187.
[9] J. E. Smay et al., Adv. Mater. 2002, 14, 1279.
[10] G. M. Gratson, M. Xu, J. A. Lewis, Nature 2004, 428, 386.
[11] C. Minas, D. Carnelli, E. Tervoort, A. R. Studart, Adv. Mater. 2016, 28, 9993.
[12] J. T. Muth et al., Proc. Natl. Acad. Sci. USA 2017, 114, 1832.
[13] Y. Lee et al. J. of Europ. Ceramic Soc. 2019, 39(14), 4358.
[14] Y. Yang et al., Internat. J. of Bioprinting 2019, 5(1), 1.
[15] A. Moreno Madrid et al., Mater. Science Engin. C 2019, 100, 631.
[16] M. P. Staiger, A. M. Pietak, J. Huadmai, G. Dias, Biomaterials 2006, 27, 1728.
[17] F. Witte et al., Biomaterials 2005, 26, 3557.
[18] F. J. O'Brien, Mater. Today 2011, 14, 88.
[19] V. Karageorgiou, D. Kaplan, Biomaterials 2005, 26, 5474.
[20] J. Wei et al., Biomaterials 2010, 31, 1260.
[21] M. P. Staiger et al., Mater. Lett. 2010, 64, 2572.
[22] T. L. Nguyen et al., Adv. Eng. Mater. 2011, 13, 872.
[23] N. Kleger, M. Cihova, K. Masania, A. Sudart, J. Löffler, Adv. Mater. 2019, 1903783.
[24] E. Johansson et al., Materials, 10(2), 2017.
[25] C. Paredes et al., J. European Ceramic Soc., 41(1):892-900, 2021.
[26] K. Wang et al., Advanced Materials, 32(25), 2020.
[27] S. Zakeri et al., Additive Manufacturing 35, 101177, 2020.
[28] J. W. Halloran, Ann. Rev. of Materials Research, 46:19-40, 2016.
[29] N. Travitzky et al., Advanced Engineering Materials 16(6), 729-754, 2014.
[30] P. F. Jacobs, Rapid Prototyping & Manufacturing: Fundamentals of Stereolithography, 1992.
[31] M. Moncada et al., J. Dairy Science 98, 5946-5954, 2015.
[32] S. Y. Song et al., Materials and Design 180, 107960, 2019.
[33] Y. De Hazan et al., J. of Colloid and Interface Science 337(1), 66-74, 2009.
[34] A. Moeck et al., “Shrinkage of UV Oligomers and Monomers” https://radtech.org/proceedings/2014/papers/Formulation/Moeck—Shrinkage of UV Oligomers and Monomers.pdf (obtained on 16 Jul. 2021).
[35] E. A. Guzzi, G. Bovone, M. W. Tibbitt, Small 2019, 15.
[36] J. Dong et al., Acta Biomater 2020, 114, 497.
[37] N. Paunovic et al., Sci Adv 2021, 7.
[38] D. W. McOwen et al., Adv Mater 2018, 30.
[39] A. Paolini, S. Kollmannsberger, E. Rank, Addit Manuf 2019, 30.
[40] Y. F. Zhang at al., Adv Mater Technol-Us 2019, 4.
[41] Z. Q. Dong at al., Nat Commun 2021, 12.
[42] N. Kleger et al., Sci Rep-Uk 2021, 11.
[43] S. Gantenbein et al., Nature 2018, 561, 226.
[44] D. G. Moore, L. Barbera, K. Masania, A. R. Studart, Nat Mater 2020, 19, 212.
[45] T. D. Ngo et al., Compos Part B-Eng 2018, 143, 172.
[46] E. Davoodi at al., Acta Biomater 2020, 117, 261.
[47] I. Capasso et al., Sci Rep-Uk 2020, 10.
[48] J. H. Shin at al., J Hazard Mater 2019, 365, 494.
[49] S. Zekoll et al., Energ Environ Sci 2018, 11, 185.
[50] S. Mohanty et al., Mat Sci Eng C-Mater 2015, 55, 569.
[51] Y. H. Jiang, Q. M. Wang, Sci Rep-Uk 2016, 6.
[52] F. Gallien, V. Gass, A. Mortensen, Materials & Design 2022, 215, 110488.
[53] L. R. Meza, S. Das, J. R. Greer, Science 2014, 345, 1322.
[54] T. A. Schaedler et al., Science 2011, 334, 962.
[55] S. Mamatha, P. Biswas, D. Das, R. Johnson, Ceram Int 2019, 45, 19577.
[56] M. Dressler et al., J Eur Ceram Soc 2009, 29, 3333.
[57] S. B. Lee et al., Biomaterials 2005, 26, 1961.
[58] Y. C. Chiu et al., Tissue Engineering Part C: Methods 2010, 16, 905.
[59] M. Schwentenwein, J. Homa, Int J Appl Ceram Tec 2015, 12, 1.
[60] M. Hartmann, M. Pfaffinger, J. Stampfl, Materials 2021, 14.
[61] R. Goodall, J. F. Despois, A. Mortensen, J Eur Ceram Soc 2006, 26, 3487.
[62] C. Hall, A. Hamilton, Mater Struct 2016, 49, 3969.
[63] B. Coppola et al., J Eur Ceram Soc 2022, 42, 2974.
[64] F. Baino et al., J Am Ceram Soc 2022, 105, 1648.
[65] H. P. Degischer at al., in Handbook of Cellular Metals, 2002, 5.

1. A photopolymerizable slurry, comprising
a plurality of particles of an inorganic salt; and
at least one polymerizable monomer or oligomer;
wherein the cation of the inorganic salt is a metal cation; wherein the anion of the inorganic salt is neither oxide nor hydroxide wherein the inorganic salt has a melting point of above 250° C. at atmospheric pressure; wherein the inorganic salt has a solubility in water above 9% w/w at room temperature; and
wherein the metal cation of the inorganic salt is selected from a group consisting of barium, beryllium, cadmium, calcium, cesium, cobalt, copper, iron, lead, magnesium, manganese, nickel, potassium, rubidium, silver, and sodium.

2. The photopolymerizable slurry according to claim 1, wherein the anion of the inorganic salt is selected from a group consisting of bromide, chloride, fluoride, iodide, sulfate, nitrate, nitrite, carbonate, and cyanide.

3. (canceled)

4. The photopolymerizable slurry according to claim 1, wherein the photopolymerizable slurry comprises a photoinitiator, and/or an inhibitor, and/or a diluent, and/or a sintering aid.

5. The photopolymerizable slurry according to claim 1, wherein the inorganic salt particles are coated or functionalized with a dispersant.

6. A method for manufacturing a photopolymerizable slurry, the method comprising the steps:
a) providing a plurality of particles of an inorganic salt in the form of a powder; wherein said inorganic salt has a metal cation selected from a group consisting of barium, beryllium, cadmium, calcium, cesium, cobalt, copper, iron, lead, magnesium, manganese, nickel, potassium, rubidium, silver, and sodium, and an anion that is neither oxide nor hydroxide; wherein the inorganic salt has a melting point of above 250° C. at atmospheric pressure; and wherein the inorganic salt has a solubility in water above 9% w/w at room temperature;
b) providing at least one radiation curable monomer; the at least one radiation curable monomer being in the liquid phase; and
c) adding the inorganic salt particles to the liquid composition and mixing the inorganic salt particles with the liquid composition; obtaining a photopolymerizable slurry.

7. The method according to claim 6, wherein the inorganic salt particles are produced by
milling an amount of inorganic salt together with a dispersant in a liquid phase, thereby obtaining a dispersion of inorganic salt particles coated or functionalized with the dispersant; and
removing the liquid phase.

8. The method according to claim 6, wherein a photo initiator, and/or an inhibitor, and/or a diluent, and/or a sintering aid are provided.

9. A method for manufacturing a sintered ceramic article, the method comprising the steps:
a) providing a photopolymerizable slurry according to claim 1;
b) selectively curing the photopolymerizable slurry to obtain a green body article;
c) debinding the green body article to obtain a binderless body article; and
d) sintering the binderless body article to obtain a sintered ceramic article.

10. The method according to claim 9, wherein the selective curing of the photopolymerizable slurry to obtain a green body article is carried out within an additive manufacturing process.

11. (canceled)

12. (canceled)

13. (canceled)

14. A method for manufacturing cast articles, the method comprising
a) providing a first template mold (1); wherein the first template mold comprises a sintered ceramic article manufactured with a method according to claim 9;
b) providing a second mold (2, 2′, 2″); wherein the second mold comprises a compartment into which said first template mold can be placed;
c) mounting the first template mold into the compartment of the second mold, thereby obtaining an operative mold (1, 2, 2′, 2″);
d) casting a fluid casting material (3, 3′, 3″) into said operative mold to obtain after solidification of said casting material an infiltrated template mold (8) comprising a solid article (9) that is at least partially located within the first template mold (1); and
e) separating said solid article from the first template mold by dissolving the sintered ceramic article of the first template mold with a suitable solvent, for example water.

15. The method according to claim 14, wherein the solid article and the first template mold are removed from the second mold before dissolving the sintered ceramic article of the first template mold.

16. The method according to claim 14, wherein the solid article is positively locked in the first template mold.

17. The method according to claim 14, wherein the second mold is a permanent mold or a lost mold.

18. The method according to claim 14, wherein a metallic material or a polymer material or a ceramic material or a composite material is used as the casting material.

19. (canceled)

20. (canceled)
